(19) United States
(12) Patent Application Publication
(10) Pub. No.: US 2025/0166178 A1
(43) Pub. Date: May 22, 2025

(54) TECHNIQUES FOR TERTIARY LYMPHOID STRUCTURE (TLS) DETECTION

(71) Applicant: BostonGene Corporation, Waltham, MA (US)

(72) Inventors: Daniil Dymov, Yerevan (AM); Vladimir Kushnarev, Yerevan (AM); Nadezhda Lukashevich, Yerevan (AM); Viktor Svekolkin, Yerevan (AM); Aleksandr Sarachakov, Altai Territory (RU)

(73) Assignee: BostonGene Corporation, Waltham, MA (US)

(21) Appl. No.: 18/918,401

(22) Filed: Oct. 17, 2024

Related U.S. Application Data
(60) Provisional application No. 63/600,991, filed on Nov. 20, 2023.

(51) Int. Cl. G06T 7/00; G06T 5/20; G06T 7/13; G06T 7/62; G06V 10/46; G16H 20/00

(52) U.S. Cl. CPC G06T 7/0012; G06T 5/20; G06T 7/13; G06T 7/62; G06V 10/46; G16H 20/00; G06T 2207/20084; G06T 2207/30024; G06T 2207/30096

(57) ABSTRACT

Described herein are techniques for identifying at least one tertiary lymphoid structure (TLS) in an image of tissue. In some embodiments, the techniques include: obtaining a TLS mask indicating, for each particular pixel of multiple pixels of the image, a value indicative of a likelihood that the particular pixel is part of the at least one TLS; processing at least a portion of the image using a trained neural network model to obtain a tumor infiltrating lymphocyte (TIL) mask indicating, for each particular pixel of pixels of at least the portion of the image, a value indicative of a likelihood that the particular pixel is part of a TIL; identifying boundaries of the at least one TLS using the TLS mask and the TIL mask; and identifying a characteristic of the at least one TLS using the boundaries of the at least one TLS.

### CROSS-REFERENCE TO RELATED APPLICATIONS

This application claims the benefit of priority under 35 U.S.C. 119 to U.S. Provisional Application 63/600,991, filed on Nov. 20, 2023, titled “Techniques for Tertiary Lymphoid Structure (TLS) Detection”, which is incorporated by reference herein in its entirety.

### BACKGROUND

Transcriptomic and morphological features of the tumor microenvironment (TME) can serve as biomarkers for clinical decision making by providing prognostic information and predicting response to specific therapies. Among these features are tertiary lymphoid structures (TLSs) and tumor infiltrating lymphocytes (TIL). TLSs are organized aggregates of immune cells. TILs are lymphocytes that exert anti-tumor effects and inhibit tumor growth.

### SUMMARY

Some aspects provide for a method for identifying at least one tertiary lymphoid structure (TLS) in an image of tissue previously-obtained from a subject, the method comprising: using at least one computer hardware processor to perform: obtaining a TLS mask indicating, for each particular pixel of multiple pixels of the image, a respective numeric value indicative of a likelihood that the particular pixel is part of the at least one TLS; processing at least a portion of the image using a trained neural network model to obtain a tumor infiltrating lymphocyte (TIL) mask indicating, for each particular pixel of pixels of at least the portion of the image, a respective numeric value indicative of a likelihood that the particular pixel is part of a TIL, wherein the trained neural network model is trained to predict, for the particular pixel, the respective numeric value indicative of the likelihood that the particular pixel is part of the TIL; identifying boundaries of the at least one TLS in the image using the TLS mask and the TIL mask; and identifying one or more characteristics of the at least one TLS using the boundaries of the at least one TLS in the image.

Some aspects provide for a system, comprising: at least one computer hardware processor; and at least one non-transitory computer-readable storage medium storing processor-executable instructions that, when executed by the at least one computer hardware processor, causes the at one computer hardware processor to perform a method for identifying at least one tertiary lymphoid structure (TLS) in an image of tissue previously-obtained from a subject, the method comprising: obtaining a TLS mask indicating, for each particular pixel of multiple pixels of the image, a respective numeric value indicative of a likelihood that the particular pixel is part of the at least one TLS; processing at least a portion of the image using a trained neural network model to obtain a tumor infiltrating lymphocyte (TIL) mask indicating, for each particular pixel of pixels of at least the portion of the image, a respective numeric value indicative of a likelihood that the particular pixel is part of a TIL, wherein the trained neural network model is trained to predict, for the particular pixel, the respective numeric value indicative of the likelihood that the particular pixel is part of the TIL; identifying boundaries of the at least one TLS in the image using the TLS mask and the TIL mask; and identifying one or more characteristics of the at least one TLS using the boundaries of the at least one TLS in the image.

Some aspects provide for at least one non-transitory computer-readable storage medium storing processor-executable instructions that, when executed by at least one computer hardware processor, causes the at least one computer hardware processor to perform a method for identifying at least one tertiary lymphoid structure (TLS) in an image of tissue previously-obtained from a subject, the method comprising: obtaining a TLS mask indicating, for each particular pixel of multiple pixels of the image, a respective numeric value indicative of a likelihood that the particular pixel is part of the at least one TLS; processing at least a portion of the image using a trained neural network model to obtain a tumor infiltrating lymphocyte (TIL) mask indicating, for each particular pixel of pixels of at least the portion of the image, a respective numeric value indicative of a likelihood that the particular pixel is part of a TIL, wherein the trained neural network model is trained to predict, for the particular pixel, the respective numeric value indicative of the likelihood that the particular pixel is part of the TIL; identifying boundaries of the at least one TLS in the image using the TLS mask and the TIL mask; and identifying one or more characteristics of the at least one TLS using the boundaries of the at least one TLS in the image.

Embodiments of any of the above aspects may have one or more of the following features.

In some embodiments, identifying the boundaries of the at least one TLS in the image comprises: identifying the boundaries based on an overlap between the TLS mask and the TIL mask.

In some embodiments, identifying the boundaries based on the overlap between the TLS mask and the TIL mask comprises identifying one or more pixels for which the TIL mask indicates a respective one or more numeric values that are greater than or equal to a first threshold and for which the TLS mask indicates a respective one or more numeric values that are greater than or equal to a second threshold.

In some embodiments, identifying the one or more pixels for which the TLS mask indicates the respective one or more numeric values that are greater than or equal to the second threshold comprises: generating a binary version of the TLS mask; and identifying contours of at least one portion of the image by applying a border-following algorithm to the binary version of the TLS mask, wherein the one or more pixels are positioned within the contours of the at least one portion of the image.

Some embodiments further comprise: obtaining a set of sub-images of the image of the tissue, wherein processing the image using the trained neural network model to obtain the TIL mask comprises: processing the set of sub-images using the trained neural network model to obtain a respective set of sub-image masks, each sub-image mask in the set of sub-image masks indicating, for a respective sub-image, a respective numeric value indicative of a likelihood that pixels in the respective sub-image are part of a TIL; and generating the TIL mask using the set of sub-image masks corresponding to the set of sub-images of the image of the tissue.

In some embodiments, the set of sub-images comprises between 10 and 1,000 sub-images.

In some embodiments, the trained neural network model is a TIL neural network model, and wherein obtaining the TLS mask comprises processing the image using a TLS neural network model to obtain the TLS mask.

Some embodiments further comprise: obtaining a set of overlapping sub-images of the image of the tissue, wherein processing the image using the TLS neural network model to obtain the TLS mask comprises: processing the set of overlapping sub-images using the TLS neural network model to obtain a respective set of pixel-level sub-image masks, each pixel-level sub-image mask in the set of pixel-level sub-image masks indicating, for each particular pixel of multiple individual pixels in a respective particular sub-image, a respective probability that the particular pixel is part of a TLS; and generating the TLS mask using the set of pixel-level sub-image masks corresponding to the set of overlapping sub-images of the image of the tissue.

In some embodiments, the set of overlapping sub-images comprises between 10 and 10,000 sub-images, each sub-image in the set of overlapping sub-images overlapping with at least one other sub-image in the set of overlapping sub-images.

In some embodiments, identifying the boundaries of the at least one TLS in the image comprises: identifying respective boundaries for a plurality of TLSs in the image; and applying a filter to the respective boundaries identified for the plurality of TLSs in the tissue to obtain the boundaries of the at least one TLS in the image.

In some embodiments, applying the filter to the respective boundaries identified for the plurality of TLSs in the image comprises, for each particular TLS of the plurality of TLSs: determining a size of the particular TLS in the image; determining whether the size is greater than or equal to a threshold; and filtering out the respective boundaries of the particular TLS after determining that the size is not greater than or equal to the threshold.

In some embodiments, identifying the one or more characteristics of the at least one TLS comprises identifying at least one characteristic selected from the group consisting of: a number of TLSs in at least the portion of the image, the number of TLSs in at least the portion of the image normalized by an area of at least the portion of the image, a total area of TLSs in at least the portion of the image, the total area of the TLSs in at least the portion of the image normalized by the area of at least the portion of the image, median area of TLSs in at least the portion of the image, and the median area of the TLSs in at least the portion of the image normalized by the area of at least the portion of the image.

Some embodiments further comprise: identifying boundaries of at least one TIL in the image using the TIL mask; and identifying one or more characteristics of the at least one TIL using the boundaries of the at least one TIL.

In some embodiments, identifying the one or more characteristics of the at least one TIL comprises determining a total area of TILs in at least the portion of the image and/or determining the total area of the TILs in at least the portion of the image normalized by an area of at least the portion of the image.

In some embodiments, identifying the boundaries of the at least one TLS in the image comprises identifying the boundaries of the at least one TLS using the TLS mask, the TIL mask, and at least one other feature in the image.

In some embodiments, the at least one other feature in the image comprises at least one feature selected from the group consisting of: tissue in the image, tumor tissue in the image, and non-tumor tissue in the image.

In some embodiments, identifying the one or more characteristics of the at least one TLS comprises identifying a total area of the at least one TLS in the tumor tissue in the image and/or identifying a number of the at least one TLS in the tumor tissue in the image.

In some embodiments, identifying the one or more characteristics of the at least one TLS comprises identifying a total area of the at least one TLS in the non-tumor tissue in the image and/or identifying a number of the at least one TLS in the non-tumor tissue in the image.

Some embodiments further comprise: identifying boundaries of at least one TIL in the image using the TIL mask; and identifying one or more characteristics of the at least one TIL using the boundaries of the at least one TIL, the identifying comprising identifying an area of the at least one TIL in the tumor tissue in the image and/or identifying an area of the at least one TIL in the non-tumor tissue in the image.

In some embodiments, the image of the tissue is a whole slide image (WSI).

In some embodiments, the tissue comprises hematoxylin-eosin-stained tissue.

In some embodiments, the image is a three-channel image comprising at least 10,000 by 10,000 pixel values per channel.

In some embodiments, the image is a three-channel image comprising at least 50,000 by 50,000 pixel values per channel.

In some embodiments, the image is a three-channel image comprising at least 100,000 by 100,000 pixel values per channel.

In some embodiments, the trained neural network model comprises at least 10 million, at least 25 million, at least 50 million, or at least 100 million parameters.

In some embodiments, the trained neural network model comprises: a deconvolution neural network portion; an adapter neural network portion having an input coupled to an output of the deconvolution neural network portion; and a classification neural network portion having an input coupled to an output of the adapter neural network portion.

In some embodiments, the classification neural network portion comprises a convolutional layer, a plurality of inverted residual layers, and a classifier.

In some embodiments, the classification neural network portion further comprises a plurality of fused inverted residual layers.

In some embodiments, the trained neural network model is a TIL neural network model, and obtaining the TLS mask comprises processing the image using a TLS neural network model.

In some embodiments, the TLS neural network model comprises at least 10 million, at least 25 million, at least 50 million, or at least 100 million parameters.

In some embodiments, the TLS neural network model comprises an encoder sub-model, a decoder sub-model, and an auxiliary classifier sub-model.

In some embodiments, the subject has, is suspected of having, or is at risk of having cancer.

In some embodiments, the cancer is non-small cell lung cancer (NSCLC).

In some embodiments, the NSCLC is lung adenocarcinoma (LUAD).

In some embodiments, the cancer is breast cancer.

In some embodiments, the cancer is lung adenocarcinoma, breast cancer, cervical squamous cell carcinoma, lung squamous cell carcinoma, head and neck squamous cell carcinoma, gastric adenocarcinoma, colorectal adenocarcinoma, liver adenocarcinoma, pancreatic adenocarcinoma, or melanoma.

Some embodiments further comprise: determining, based on the one or more characteristics of the at least one TLS, to administer an immunotherapy to the subject.

Some embodiments further comprise: administering the immunotherapy to the subject.

In some embodiments, administering the immunotherapy to the subject comprises administering pembrolizumab, nivolumab, atezolizumab, or durvalumab.

### BRIEF DESCRIPTION OF DRAWINGS

Various aspects and embodiments of the disclosure provided herein are described below with reference to the following figures. The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1A and FIG. 1B are diagrams of illustrative techniques for identifying at least one tertiary lymphoid structure (TLS) in an image of tissue, according to some embodiments of the technology described herein.

FIG. 2 is a block diagram of an example system 200 for identifying at least one TLS in an image of tissue, according to some embodiments of the technology described herein.

FIG. 3A is a flowchart of an illustrative process 300 for identifying at least one TLS in an image of tissue, according to some embodiments of the technology described herein.

FIG. 3B is a flowchart of an illustrative process 330 for generating a TLS mask for an image of tissue, according to some embodiments of the technology described herein.

FIG. 3C is a flowchart of an illustrative process 340 for generating a tumor infiltrating lymphocyte (TIL) mask for an image of tissue, according to some embodiments of the technology described herein.

FIGS. 4A-4H show an example of identifying at least one TLS in an image of tissue, according to some embodiments of the technology described herein.

FIGS. 5A-5H show an example of identifying at least one TIL in an image of tissue, according to some embodiments of the technology described herein.

FIG. 6A shows an example of using neural network models to generate a TIL mask and a TLS mask, according to some embodiments of the technology described herein.

FIG. 6B is a flowchart of an illustrative process 660 for training a neural network model to identify at least one TIL in an image of tissue, according to some embodiments of the technology described herein.

FIG. 7 is an example of using a TLS neural network model and TIL neural network model to identify at least one TLS in an image of tissue, according to some embodiments of the technology described herein.

FIG. 8A is an example plot showing that overall survival improves when the density of TLSs in the tissue is greater than a threshold, according to some embodiments of the technology described herein.

FIG. 8B is an example plot showing that overall survival improves when the percentage of TILs in the tissue is greater than a threshold, according to some embodiments of the technology described herein.

FIG. 8C-8E are example plots showing that the density of TLSs and presence of TILs are independent positive prognostic factors of overall survival in subtypes of breast cancer, according to some embodiments of the technology described herein.

FIG. 9 is a schematic diagram of an illustrative computing device with which aspects described herein may be implemented.

### DETAILED DESCRIPTION

The inventors have developed techniques for identifying tertiary lymphoid structures (TLSs) in images of tissue obtained from a subject having, suspected of having, or at risk of having cancer. In some embodiments, the techniques include: (a) obtaining a TLS mask indicating, for each particular pixel of multiple pixels of an image, a respective numeric value indicative of a likelihood that the particular pixel is part of the at least one TLS, (b) obtaining a tumor infiltrating lymphocyte (TIL) mask indicating, for each particular pixel of pixels of at least a portion of the image, a respective numeric value indicating a likelihood that the particular pixel is part of a TIL, (c) identifying boundaries of the at least one TLS in the image using the TLS mask and the TIL mask, and (d) identifying one or more characteristics of the at least one TLS using the boundaries of the at least one TLS in the image. In some embodiments, the TLS and TIL masks may be obtained by applying trained machine learning models (e.g., trained neural networks) to an image or images of tissue obtained from the subject. In some embodiments, the identified characteristic(s) may be used to recommend a therapy (e.g., an immunotherapy) to be administered to the subject.

As used herein, “tertiary lymphoid structure” or “TLS” refers to ectopic lymphoid organs that develop in non-lymphoid tissues at sites of chronic inflammation, including tumors. Structural features of TLS have been described, for example by Sautés-Friedman et al., Nature Reviews Cancer volume 19, pp. 307-325 (2019). In some embodiments, the presence of TLS in a biological sample obtained from a subject is indicative of the subject having a better prognosis (e.g., relative to subjects not having TLS), and/or responding efficiently to immunotherapies (e.g., relative to subjects not having TLS), for example as described by Dieu-Nosjean et al. Immunol Rev. 2016 May; 271(1):260-75. doi: 10.1111/imr.12405.

As used herein, “tumor infiltrating lymphocytes” or “TILs” refer to lymphocytes within and around cancer cells. In some embodiments, TILs are lymphocytes within and around a solid tumor. Examples of TILs are described by Whiteside, T. (“Tumor-infiltrating lymphocytes and their role in solid tumor progression.” Interaction of immune and cancer cells. Cham: Springer International Publishing, 2022. 89-106), which is incorporated by reference herein in its entirety. In some embodiments, the presence of TILs in a biological sample obtained from a subject is indicative of the subject having a better prognosis (e.g., relative to subjects not having TILs) and/or responding to immunotherapies (e.g., relative to subjects not having TILs).

The presence of TLSs and TILs in tumor tissue have been shown to be associated with prolonged patient survival and a positive therapeutic response to immunotherapy. Accordingly, tumor tissue may be analyzed for the presence of TLSs and TILs, and characteristics of the identified TLSs and TILs may be used to predict how a patient will respond to a particular therapy, to diagnose the patient, and/or to estimate patient survival. For example, tumor tissue having a greater number of TLSs and/or a greater area occupied TLSs may indicate that a patient will have prolonged survival and may respond positively to a particular immunotherapy. Additionally, tumor tissue having a greater proportion of TILs may suggest that patient will have prolonged survival.

Conventionally, TLS and TIL identification is manually performed by pathologists. For example, the pathologist may obtain an image of tissue, visually assess the image, and manually label regions of the image that they identify as including a TLS and/or TILs. There are multiple problems associated with such conventional techniques. One problem is that identifying TLSs and TILs is a subjective procedure that results in issues of reproducibility caused by different pathologists making different decisions about how to label an image. Accordingly, treatment recommendations and/or survival outcomes estimated based on such data may also differ. Another problem with conventional techniques is that the manual analysis of such data is very inefficient. Manually labelling such image data is extremely time-consuming, leading to high costs, which in turn affects the quality of the data processing results or the study overall.

Recently, there have been developments towards automated approaches for detecting the presence of TLSs in tumor tissue. In particular, machine learning techniques have been developed to automate the process of detecting the presence of TLSs in tumor tissue. While such techniques are promising, the inventors have recognized that their accuracy and performance can be improved by accounting for the presence of TILs in the tissue.

Accordingly, the inventors have developed techniques that further improve the existing automated TLS identification techniques. In some embodiments, the techniques involve (a) obtaining a TLS mask indicating, for each particular pixel of multiple pixels of the image, a respective numeric value indicative of a likelihood that the particular pixel is part of the at least one TLS; (b) processing at least a portion of the image using a trained neural network model to obtain a tumor infiltrating lymphocyte (TIL) mask indicating, for each particular pixel of pixels of at least the portion of the image, a respective numeric value indicative of a likelihood that the particular pixel is part of a TIL; (c) identifying boundaries of the at least one TLS in the image using the TLS mask and the TIL mask; and (d) identifying one or more characteristics of the at least one TLS using the boundaries of the identified at least one TLS in the image. In some embodiments, the trained neural network is trained to predict, for the particular pixel, the respective numeric value indicative of the likelihood that the particular pixel is part of the TIL. In some embodiments, the identified characteristics of the identified TLS(s) may be used to recommend a therapy (e.g., an immunotherapy) to be administered to the subject.

The techniques developed by the inventors are an improvement over the conventional automated TLS identification techniques because they account not only for TLSs, but also for the presence of TILs in the tissue. Detecting and quantifying the presence of TILs in the tissue (e.g., tumor tissue) helps to assess the level of immune cell infiltration in the tissue, as well as the level of overall immune response. For example, presence of both TLSs and TILs in the tissue, may be prognostic of longer overall survival and/or a positive response to certain types of therapies. However, absence (or an insufficient amount) of one or both of TLSs and TILs in the tissue, may be prognostic of shorter overall survival and/or non-responsiveness to certain types of therapies. Accordingly, by accounting for the presence of both features (e.g., TLSs and TILs), the comprehensive techniques developed by the inventors can be used to predict survival outcomes and therapeutic response more accurately, making the techniques more reliable for making treatment decisions and informing diagnoses.

Another aspect of the techniques developed by the inventors that contributes to the improvements over existing TLS identification techniques is the use of one or more neural networks to identify TLS(s) in an image of tissue. For example, a trained neural network may be used to process the image to obtain a mask (e.g., a TIL mask) indicating likelihoods that pixels in the image are part of at least one TIL. In some embodiments, the TIL mask can be used to identify TILs in the image. Additionally, the TIL mask can be used to identify at least one TLS in the image. For example, the TIL mask and a TLS mask obtained for the image can be used to identify the at least one TLS. The TLS may indicate likelihoods that pixels in the image are part of at least one TLS. The TLS mask may be obtained according to any suitable techniques, as aspects of the technology described herein are not limited in this respect. For example, a trained neural network may be used to process the image to obtain the TLS mask. The use of the one or more neural networks for identifying different features in an image of tissue is more efficient than the conventional techniques which rely on manual annotation of such features.

In some embodiments, at least one TLS in an image is identified based on an intersection between features in the image. For example, the at least one TLS may be identified based on an intersection between at least one TIL indicated by a TIL mask and at least one TLS indicated by a TLS mask. The intersection between the TLS and the TIL can be used to quantify characteristics of one or more TLSs that are infiltrated with TILs. As described herein, the presence of one or both TLSs and TILs in tissue may be prognostic of longer overall survival and/or a positive response to certain types of therapies (see the “Examples” section). Accordingly, identifying the presence of both features in the tissue and quantifying characteristics of the combination of said features can be used to more accurately predict overall survival and therapeutic response.

Additionally, the at least one TLS may be identified based on an intersection between at least one TIL, at least one TLS, and at least one other feature. For example, the at least one other feature may include tissue (e.g., as opposed to background or image artifacts), tumor tissue, non-tumor tissue, borderline tumor tissue (e.g., non-tumor tissue within a threshold distance of the tumor tissue), or any other suitable feature, as aspects of the technology described herein are not limited in this respect. In some embodiments, identifying an intersection between the at least one TIL, at least one TLS, and tissue may be used to more accurately evaluate immune response in the tissue by excluding regions of the image that do not correspond to tissue. In some embodiments, identifying intersections between the at least one TIL, at least one TLS, and different types of tissue (e.g., tumor, borderline tumor, and non-tumor tissue) can be used to evaluate and compare differences in immune response across the different types of tissue.

Following below are descriptions of various concepts related to, and embodiments of, techniques for identifying at least one TLS in an image of tissue. It should be appreciated that various aspects described herein may be implemented in any of numerous ways, as techniques are not limited to any particular manner of implementation. Examples of details of implementations are provided herein solely for illustrative purposes. Furthermore, the techniques disclosed herein may be used individually or in any suitable combination, as aspects of the technology described herein are not limited to the use of any particular technique or combination of techniques.

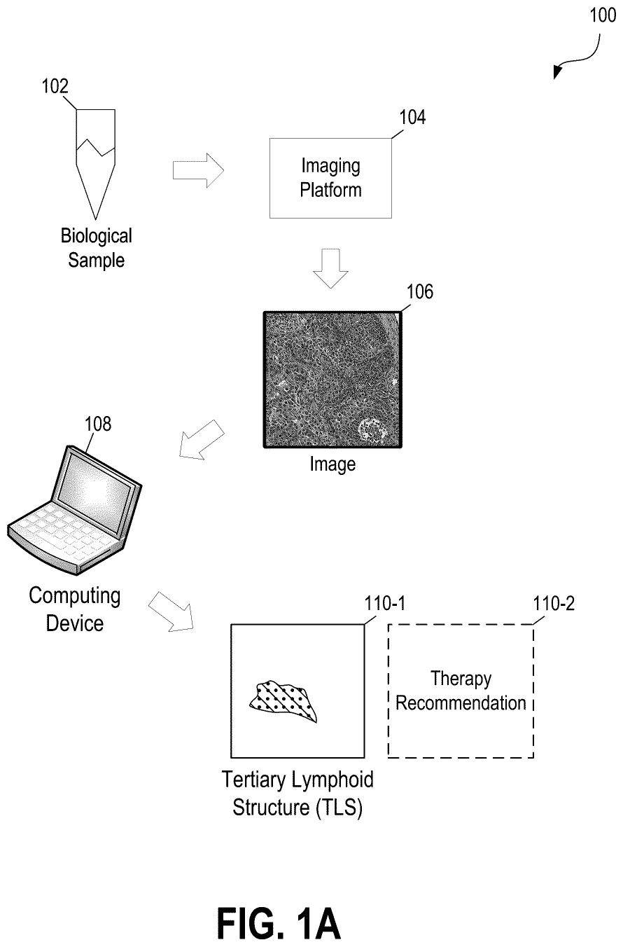

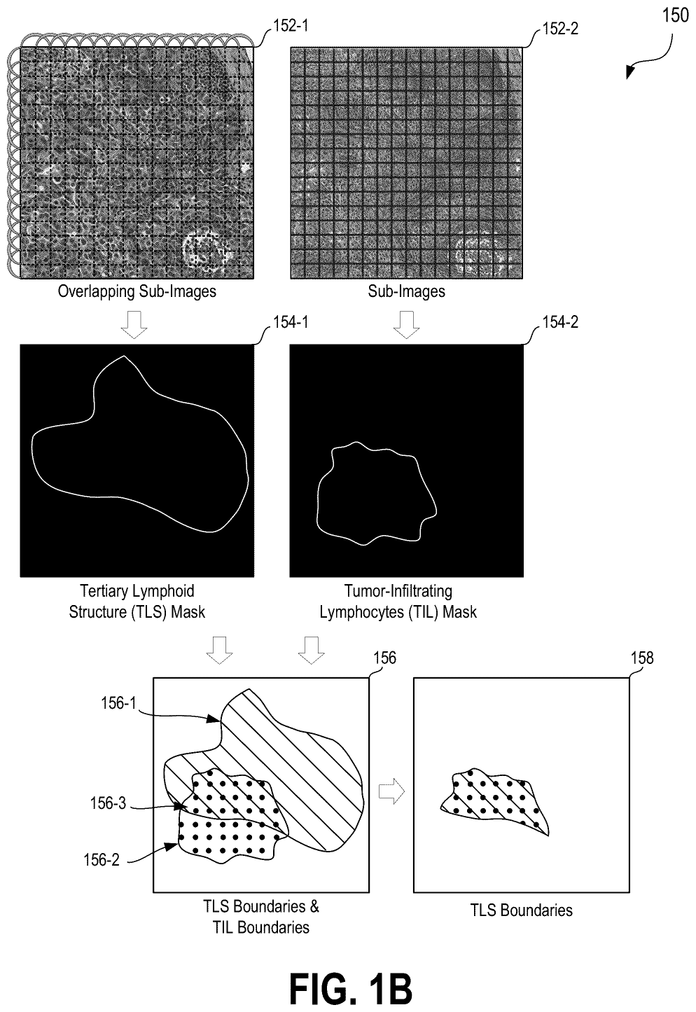

FIG. 1A is a diagram of an illustrative technique 100 for identifying at least one tertiary lymphoid structure (TLS) in an image of tissue, according to some embodiments of the technology described herein. Technique 100 includes obtaining an image 106 of tissue and processing the image 106 using computing device 108 to identify at least one TLS 110-1 and/or to obtain a therapy recommendation 110-2 for a subject. In some embodiments, the image 106 is obtained or was previously obtained by imaging a biological sample 102 using an imaging platform 104.

In some embodiments, aspects of the illustrative technique 100 may be implemented in a clinical or laboratory setting. For example, aspects of the illustrative technique 100 may be implemented on a computing device 108 that is located within the clinical or laboratory setting. In some embodiments, the computing device 108 may obtain the image 106 from an imaging platform 104 co-located with the computing device 108 within the clinical or laboratory setting. For example, the computing device 108 may be included in the imaging platform 104. In some embodiments, the computing device 108 may indirectly obtain the image 106 from the imaging platform 104 located externally from or co-located with the computing device 108. For example, the computing device 108 may obtain the image 106 via at least one communication network, such as the Internet or any other suitable communication network(s), as aspects of the technology described herein are not limited in this respect.

In some embodiments, aspects of the illustrative technique 100 may be implemented in a setting that is located externally from a clinical or laboratory setting. In this case, the computing device 108 may indirectly obtain the image 106 from the imaging platform 104 located within or externally to a clinical or laboratory setting. For example, the image 106 may be provided to the computing device 108 via at least one communication network such as the Internet or any other suitable communication network(s), as aspects of the technology described herein are not limited in this respect.

In some embodiments, technique 100 includes obtaining a biological sample 102 from a subject. In some embodiments, the biological sample 102 was previously obtained from the subject. The biological sample 102 may be obtained from a subject having, suspected of having, or at risk of having cancer and/or any immune-related disease. The biological sample 102 may be obtained by performing a biopsy or by obtaining a blood sample, a salivary sample, or any other suitable biological sample from the subject. The biological sample 102 may include diseased tissue (e.g., cancerous), and/or healthy tissue. In some embodiments, prior to imaging, the biological sample 102 may be stained using a histological stain. For example, the biological sample 102 may be stained using a hematoxylin and eosin (H&E) stain, Masson triple or trichrome stain, an elastic fiber stain, a silver stain, a period acid Schiff (PAS) stain or any other suitable type of stain. In some embodiments, the origin or preparation methods of the biological sample may include any of the embodiments described herein including with respect to the “Biological Samples” section.

The imaging platform 104 may include any instrument, device, and/or system suitable for imaging a biological sample (e.g., tissue in a slide), as aspects of the technology are not limited in this respect. For example, the imaging platform 104 may include a whole slide imaging (WSI) scanner, a digital microscope, or any other suitable instrument, device, and/or system for pathology imaging of tissue. In some embodiments, the biological sample 102 may be prepared according to the manufacturer's protocol associated with the imaging platform 104.

In some embodiments, the imaging platform 104 may be configured to store an image at multiple resolutions to facilitate retrieval of image data at any suitable resolution. For example, the imaging platform 104 may be configured to store image data at the highest resolution as captured by the imaging platform 104 and at one or more resolutions lower than the highest resolution. For example, image 106 may include image data at the highest resolution captured by imaging platform 104, or it may include image data at a lower resolution (e.g., a 4× downscale of the highest resolution).

The image 106 may be a single-channel image or a multi-channel image (e.g., a 3-channel RGB image). Though it should be appreciated that the image 106 may comprise pixel values for any suitable number of channels depending on how imaging is performed, and the imaging platform used for same.

In some embodiments, the image 106 may include a whole slide image (WSI). The image 106 may have any suitable dimensions, as aspects of the technology are not limited in this respect. For example, the image 106 may have at least 100,000×100,000 pixel values per channel, 75,000×75,000 pixel values per channel, 50,000×50,000 pixel values per channel, 25,000×25,000 pixel values per channel, 10,000×10,000 pixel values per channel, 5,000×5,000 pixel values per channel or any other suitable number of pixels per channel. The dimensions of image 106 may be within any suitable range such as, for example, 50,000-500,000×50,000-500,000 pixel values per channel, 25,000-1 million×25,000-1 million pixel values per channel, 5,000-2 million×5,000-2 million pixel values per channel, or any other suitable range within these ranges.

In some embodiments, computing device 108 is used to process image 106. The computing device 108 may be operated by a user such as a doctor, clinician, researcher, patient, and/or any other suitable individual or entity. For example, the user may provide the image 106 as input to the computing device 108 (e.g., by uploading a file), and/or may provide user input specifying processing or other methods to be performed using the image 106.

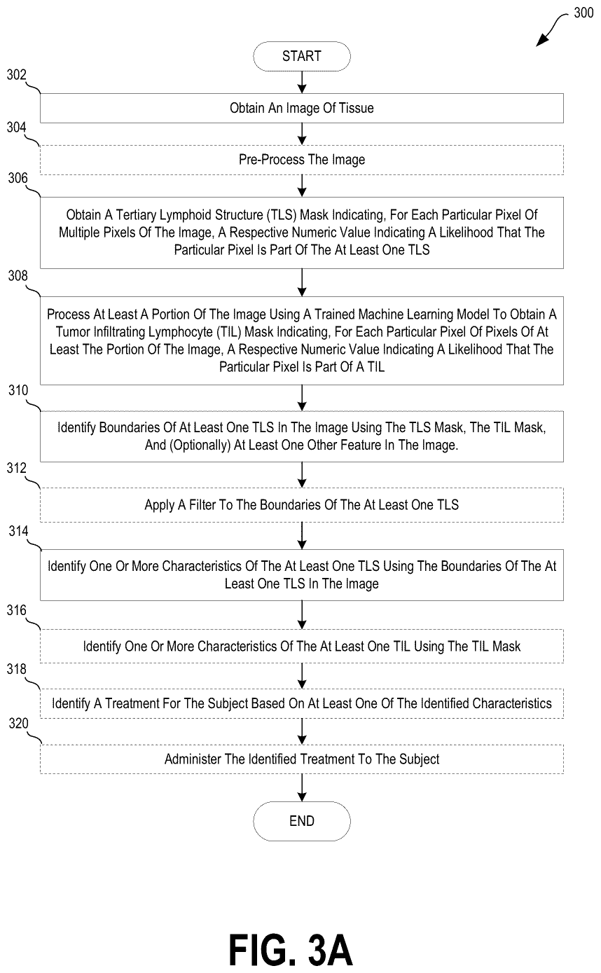

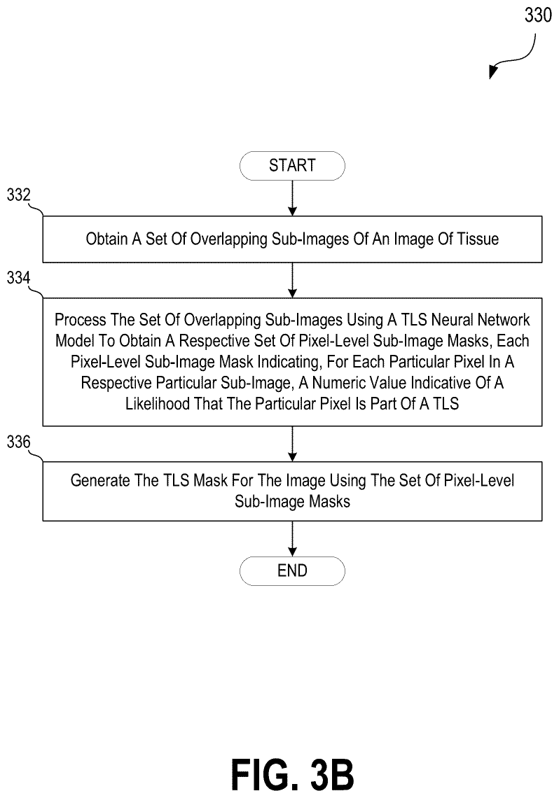

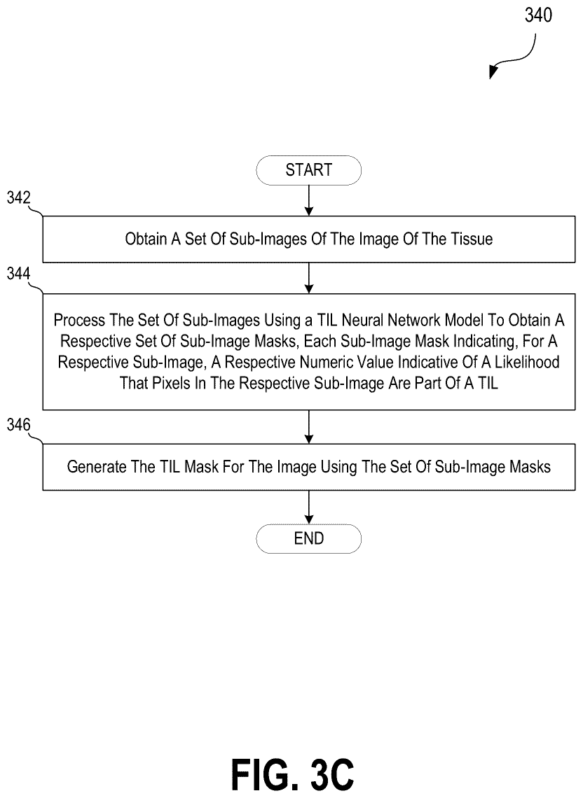

In some embodiments, computing device 108 includes software configured to perform various functions with respect to the image 106. An example of computing device 108 including such software is described herein including at least with respect to FIG. 1B. In some embodiments, software on computing device 108 may be configured to process at least a portion of the image 106 (e.g., the whole image or less than the whole image) to identify at least one TLS in the portion of the image 106. In some embodiments, this may include: (a) obtaining a TLS mask indicating likelihoods that pixels of the image are part of at least one TLS, (b) processing at least a portion of the image using a trained neural network model to obtain a TIL mask indicating likelihoods that pixels of the image are part of a TIL, (c) identifying boundaries of the at least one TLS in the image using the TLS mask, the TIL mask, and (optionally) at least one other feature in the image (e.g., tumor tissue, non-tumor tissue, tissue without artifacts, etc.); and (d) identifying one or more characteristics of the at least one TLS using the boundaries of the at least one TLS in the image. Example techniques for identifying at least one TLS in an image are described herein including at least with respect to FIG. 1B and FIGS. 3A-3C.

In some embodiments, the boundaries of the TLS may be used to distinguish between regions of the image 106 that include one or more TLSs and regions of the image that do not include any TLS. Such boundaries may be used to distinguish between different TLSs in the image 106.

In some embodiments, the identified boundaries of the TLS may be used to identify one or more characteristics of the TLS. A characteristic may include any suitable characteristic of TLS(s), as aspects of the technology are not limited in this respect. As non-limiting examples, the features may include a number of TLSs in at least the portion of the image, the number of TLSs in at least a portion of the image normalized by area of the portion of the image, a total area of TLSs in at least a portion of the image, the total area of TLSs in at least a portion of the image normalized by the area of at the portion of the image, median area of TLSs in at least a portion of the image, and the median area of TLSs in at least a portion of the image normalized by the area of the portion of the image.

In some embodiments, software on the computing device 108 may additionally or alternatively be configured to identify boundaries of TIL(s) in the image 106 and to use the identified boundaries to determine one or more characteristics of the TILs. A characteristic may include any suitable characteristic of TIL(s), as aspects of the technology are not limited in this respect. As non-limiting examples, the features may include a total area of TILs in at least a portion of the image, the total area of TILs in at least a portion of the image normalized by the area of at the portion of the image, median area of TILs in at least a portion of the image, and the median area of TILs in at least a portion of the image normalized by the area of the portion of the image.

In some embodiments, software on the computing device 108 may use the identified TLS and/or TIL characteristics to generate a recommendation for treating a subject from which biological sample 102 was obtained. For example, one or more of the identified characteristics may be used as prognostic or predictive biomarkers for diagnosing the subject, predicting overall survival for the subject, and/or predicting how the subject will respond to a particular therapy. Accordingly, in some embodiments, the software on the computing device 108 may generate a recommendation for diagnosing the subject and/or for administering a particular therapy to the subject. In some embodiments, the software may be configured to predict, based on the identified characteristics, how a subject will respond to a particular therapy, and may recommend administration of the particular therapy when the subject is predicted to respond positively to the particular therapy. For example, when the number of TLSs in at least a portion of an image normalized by the area of the portion of the image is greater than a threshold, the software may be configured to recommend an immunotherapy (e.g., a checkpoint inhibitor immunotherapy) for the subject.

In some embodiments, technique 100 includes generating output(s). The output(s) may include an indication 110-1 of the identified TLSs. For example, the indication 110-1 may include one or more masks generated by processing image 106 using computing device 108 and/or a version of at least a portion of image 106 indicating the boundaries of at least one TLS and/or TIL. Additionally, or alternatively, indication 110-1 may indicate the probability that the image 106 includes or does not include at least one TLS. Additionally, or alternatively, indication 110-1 may indicate the probability that the image 106 includes or does not include at least one TIL. Additionally, or alternatively, the output(s) may include a recommendation 110-2 for administering a particular therapy (e.g., an immunotherapy such as a checkpoint inhibitor therapy) to and/or diagnosing the subject from which biological sample 102 was obtained.

In some embodiments, the output(s) are stored (e.g., in memory), displayed via a user interface, transmitted to one or more other devices, or otherwise processed using any suitable techniques, as aspects of the technology are not limited in this respect. For example, the output(s) may be displayed using a graphical user interface (GUI) of a computing device (e.g., computing device 108).

FIG. 1B is a diagram of an illustrative technique 150 for identifying at least one tertiary lymphoid structure (TLS) in an image of tissue (e.g., image 106 in FIG. 1A), according to some embodiments of the technology described herein. Technique 150 includes (a) obtaining a TLS mask 154-1 for the image, (b) obtaining a TIL mask 154-2 for the image, and (c) using the TLS mask 154-1 and TIL mask 154-2 to identify boundaries 158 of the TLS.

The TLS mask 154-1 may be obtained using any suitable techniques, as aspects of the technology described herein are not limited in this respect. For example, the TLS mask 154-1 may have been previously generated by a user and/or using a computing device. Such a TLS mask 154-1 may be obtained from a user (e.g., by the user uploading the TLS mask 154-1), from a data store, from an external device, or from any other suitable source, as aspects of the technology described herein are not limited in this respect. Additionally, or alternatively, in some embodiments, the TLS mask 154-1 may be generated using a neural network.

In some embodiments, generating a TLS mask 154-1 using a neural network model may include (a) generating, from the image, a set of multiple overlapping sub-images 152-1, (b) processing the set of overlapping sub-images using a trained neural network model to obtain a respective set of pixel-level sub-image masks, and (c) determining a pixel-level mask 154-1 for the portion of image based on the pixel-level sub-image masks. In some embodiments, a pixel-level sub-image mask may indicate, for each of multiple pixels in a respective sub-image, a numeric value indicative of the likelihood that the particular pixel is part of a TLS. Example techniques for generating a TLS mask are described herein including at least with respect to FIG. 3B.

In some embodiments, obtaining the TIL mask 154-2 for the image includes processing the image using a trained neural network model to obtain the TIL mask. Processing the image using the trained neural network model may include (a) generating, from the image, a set of sub-images (e.g., non-overlapping sub-images) 152-2, (b) processing the set of sub-images using a trained neural network model to obtain a respective set of sub-image masks, and (d) determining a mask 154-2 for at least a portion of the image based on the sub-image masks. In some embodiments, a sub-image mask may indicate, for a respective sub-image, a numeric value indicative of the likelihood that pixels in the respective sub-image are part of a TIL. Example techniques for generating a TIL mask are described herein including at least with respect to FIG. 3C.

In some embodiments, as shown in box 156, the TLS mask 154-1 and TIL mask 154-2 are used to identify boundaries 158 of at least one TLS in the image. For example, as shown in box 156, the TLS mask 154-1 may be used to identify a region 156-1. Region 156-1 may include pixels for which the TLS mask 154-1 indicates numeric values that are greater than or equal to a first threshold value. The numeric values may be indicative a likelihood that the pixels are part of a TLS. The TIL mask 154-2 may be used to identify a region 156-2. Region 156-2 may include pixels for which the TIL mask 154-2 indicates numeric values that are greater than or equal to a second threshold value. The numeric values may be indicative of a likelihood that the pixels are part of a TIL.

In some embodiments, identifying the boundaries 158 include identifying a region of overlap 156-3 between the region 156-1 and region 156-2. For example, the region of overlap 156-3 may include pixels for which the TLS mask indicates numeric values that are greater than equal to the first threshold value and for which the TIL mask indicates numeric values that are greater than or equal to the second threshold value. In some embodiments, the boundaries of the region 156-3 are identified as the boundaries 158 of the at least one TLS.

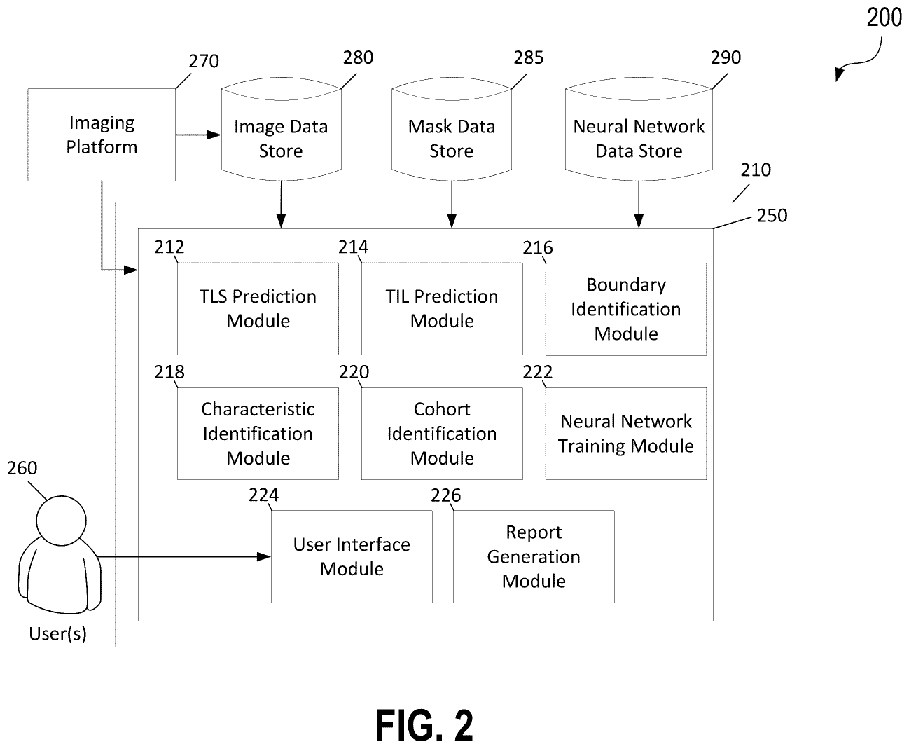

FIG. 2 is a block diagram of an example system 200 for identifying at least one TLS in an image of tissue, according to some embodiments of the technology described herein. System 200 includes computing device(s) 210 configured to have software 250 execute thereon to perform various functions in connection with identifying at least one TLS in an image of tissue. In some embodiments, the software 250 includes a plurality of modules. A module may include processor-executable instructions that, when executed by at least one computer hardware processor, cause the at least one computer hardware processor to perform function(s) of the module. Such modules are sometimes referred to herein as “software modules,” each of which includes processor-executable instructions configured to perform one or more processes, such as process 300 shown in FIG. 3A, process 330 shown in FIG. 3B, and process 340 shown in FIG. 3C.

The computing device(s) 210 may be operated by one or more user(s) 260. For example, the user(s) 260 may include one or more individuals who are treating and/or studying (e.g., doctors, clinicians, researchers, etc.) the subject. Additionally, or alternatively, user(s) 260 may include the subject. In some embodiments, the user(s) 260 may provide, as input to the computing device(s) 210 (e.g., by uploading one or more files, by interacting with a user interface of the computing device(s) 210, etc.) image(s) (e.g., image 106 in FIG. 1A). Additionally, or alternatively, the user(s) 260 may provide input specifying processing or other methods to be performed on the image(s). Additionally, or alternatively, the user(s) 260 may access results of processing the image(s). For example, user(s) 260 may access results of identifying at least one TLS in the image(s) and/or a therapy recommendation for the subject.

As shown in FIG. 2, software 250 includes multiple software modules for identifying at least one TLS in an image and/or recommending a therapy for a subject. Some software modules include a TLS prediction module 212, a TIL prediction module 214, a boundary identification module 216, a characteristic identification module 218, and a cohort identification module 220.

In some embodiments, the TLS prediction module 212 obtains image(s) (e.g., image 106 in FIG. 1A) from imaging platform 270 (e.g., imaging platform 104 in FIG. 1A), image data store 280, and/or user(s) 260 (e.g., by the user uploading the image(s)).

In some embodiments, the TLS prediction module 212 is configured to determine a probability that at least a portion of an image includes a TLS. For example, the TLS prediction module 212 may be configured to process one or more sub-images of the portion of the image using a neural network model to obtain a respective set of pixel-level sub-image masks. As described herein, a pixel level sub-image mask may indicate for each of multiple pixels in a sub-image, a numeric value indicative of a likelihood that the particular pixel is part of a TLS. In some embodiments, the TLS prediction module 212 is further configured to determine a pixel-level mask for the portion of the image based on the pixel-level sub-image masks. For example, the TLS prediction module 212 may be configured to combine the pixel-level sub-image masks to obtain the pixel-level mask for the portion of the image. Example techniques for determining a probability that a portion of an image includes a TLS are described herein including at least with respect to process 330 shown in FIG. 3B.

In some embodiments, the TIL prediction module 214 obtains image(s) (e.g., image 106 in FIG. 1A) from imaging platform 270 (e.g., imaging platform 104 in FIG. 1A), image data store 280, and/or user(s) 260 (e.g., by the user uploading the image(s)).

In some embodiments, the TIL prediction module 214 is configured to determine a probability that at least a portion of the image includes a TIL. For example, the TIL prediction module 214 may be configured to process one or more sub-images of the portion of the image using a neural network model to obtain a respective set of sub-image masks. As described herein, a sub-image mask may indicate a numeric value indicative of a likelihood that one or more pixels in a respective sub-image are part of a TIL. In some embodiments, the TIL prediction module 214 is further configured to determine a mask for the portion of the image based on the sub-image masks. Example techniques for determining a probability that a portion of an image includes a TIL are described herein including at least with respect to process 300 shown in FIG. 3A and process 340 shown in FIG. 3C.

In some embodiments, the boundary identification module 216 obtains a TLS mask from TLS prediction module 212, mask data store 285, user(s) 260 (e.g., by the user uploading the TLS mask). Additionally, or alternatively, in some embodiments, the boundary identification module 216 obtains a TIL mask from TIL prediction module 214, mask data store 285, user(s) 260 (e.g., by the user uploading the TIL mask). Additionally, or alternatively, in some embodiments, the boundary identification module 216 obtains a mask and/or indication of one or more other features in the image. For example, the boundary identification module 216 may obtain the mask and/or indication from the mask data store 285, user(s) 260 (e.g., by the user(s) uploading the mask and/or indication), and/or from a feature prediction module (not shown).

In some embodiments, the boundary identification module 216 is configured to identify boundaries of at least one TLS in at least a portion of the image. In some embodiments, identifying the boundaries includes identifying a region of overlap between the TLS mask, TIL mask, and/or a mask generated for another feature. For example, this may include (a) identifying boundaries of at least one candidate TLS using the TLS mask, (b) identifying boundaries of at least one TIL using the TIL mask, and/or (c) identifying boundaries of at least one other feature using the respective mask, then using the identified boundaries to identify a region of overlap between the candidate TLS, the TIL, and/or the other feature.

In some embodiments, identifying boundaries of at least one candidate TLS using the TLS mask may be achieved by generating a binary version of the pixel-level mask output by the TLS prediction module 212 (e.g., by binarizing the mask with respect to a threshold) and identifying contours of at least one TLS using any suitable technique, as aspects of the technology described herein are not limited in this respect. For example, the contours may be identified by applying a border-following algorithm to the binary version of the pixel-level mask.

In some embodiments, identifying boundaries of at least one candidate TIL using the TIL mask may be achieved by generating a binary version of the mask output by the TIL prediction module 214 (e.g., by binarizing the mask with respect to a threshold) and identifying contours of at least one TIL using any suitable technique, as aspects of the technology described herein are not limited in this respect. For example, the contours may be identified by applying a border-following algorithm to the binary version of the pixel-level mask.

In some embodiments, the at least one other feature includes any suitable feature such as, for example, tissue (e.g., excluding image artifacts and background), tumor tissue, non-tumor tissue, and/or borderline tissue (e.g., non-tumor tissue within a threshold distance of the tumor tissue). Tumor tissue may include tissue that includes tumor cells. For example, the tumor tissue may include greater than or equal to a threshold proportion of tumor cells. Non-tumor tissue may include tissue that includes non-tumor cells. For example, the non-tumor tissue may include less than the threshold proportion of tumor cells. Borderline tissue may include non-tumor tissue that is within a threshold distance of the tumor tissue.

In some embodiments, the characteristic identification module 218 is configured to identify one or more characteristics of the at least one TLS for which boundaries were identified. For example, the characteristic identification module 218 may use the boundaries to determine a number of TLSs in a portion of the image, an area of each TLS, and/or an area of the portion of the image that does not include any TLSs. These values may serve as characteristics and/or may be used to determine additional TLS characteristics. For example, the characteristic identification module 218 may determine the number of TLSs in at least a portion of the image normalized by area of the portion of the image, a total area of TLSs in at least a portion of the image, the total area of TLSs in at least a portion of the image normalized by the area of at least the portion of the image, median area of TLSs in at least a portion of the image, and/or the median area of TLSs in at least a portion of the image normalized by the area of the portion of the image.

Additionally, or alternatively, in some embodiments, the characteristic identification module 218 is configured to identify one or more characteristics of at least one TIL for which boundaries were identified. For example, the characteristic identification module 218 may use the boundaries of the TILs to determine an area of the portion of the image including TILs, and/or an area of the portion of the image that does not include TILs. These values may serve as characteristics and/or may be used to determine further TIL characteristics. For example, the characteristic identification module 218 may determine the area of TILs in a portion of the image normalized by the area of the portion of the image.

In some embodiments, the cohort identification module 220 uses the characteristics identified by the characteristic identification module 218 to identify a cohort for the subject. In some embodiments, a cohort corresponds to a particular therapeutic response, with a particular overall survival, and/or a particular diagnosis. In some embodiments, identifying a cohort for a subject includes comparing a value of one or more TLS characteristics to criteria associated with the cohort, and identifying the cohort for the subject when the criteria is satisfied. As a nonlimiting example, breast cancer patients having a TLS density of greater than 2 TLS/mm² have been shown to have an increased overall survival percentage over 120 months compared to breast cancer patients having a TLS density of less than 2 TLS/mm². The cohort identification module 220 may be configured to compare the determined TLS density to a threshold of 2 TLS/mm² and, upon determining that the TLS density exceeds the threshold, identify a cohort for the subject that is associated with increased overall survival percentage.

Additionally, or alternatively, in some embodiments the cohort identification module 220 is configured to identify a cohort for the subject based on characteristics of the TILs in the image. For example, the cohort identification module 220 may be configured to compare the area of TILs in a portion of the image that includes tumor tissue to the area of TILs in a portion of the image that includes non-tumor tissue to determine a degree of immune response in the tumor tissue.

In some embodiments, based on the cohort identified for the individual, the cohort identification module 220 may recommend a therapy for the subject.

In some embodiments, software 250 further includes report generation module 226. In some embodiments, report generation module 226 is configured to generate a report based on the outputs of the TLS prediction module 212, the TIL prediction module 214, the boundary identification module 216, the characteristic identification module 218, and/or the cohort identification module 220. For example, the report generation module 226 may generate a report that includes a pixel-level mask output by TLS prediction module, a mask output by the TIL prediction module, an indication of the boundaries of one or more TLSs in an image of tissue, an indication of one or more TILs, an indication of one or more characteristics of the TLSs and/or TILs, an indication of one or more cohorts identified for the subjects, and/or any other suitable information, as aspects of the technology are not limited in this respect.

In some embodiments, software 250 further includes user interface module 224. User interface module 224 may be configured to generate a graphical user interface (GUI) through which the user may provide input and view information generated by software 250. For example, in some embodiments, the user interface module 224 may be a webpage or web application accessible through an Internet browser. In some embodiments, the user interface module 224 may generate a graphical user interface (GUI) of an app executing on the user's mobile device. In some embodiments, the user interface module 224 may generate a GUI on a imaging platform, such as imaging platform 270. In some embodiments, the user interface module 224 may generate a number of selectable elements through which a user may interact. For example, the user interface module 224 may generate dropdown lists, checkboxes, text fields, or any other suitable element. In some embodiments, the user interface module 224 is configured to generate a GUI including report(s) generated by report generation module 226.

As shown in FIG. 2, system 200 also includes image data store 280, mask data store 285, and neural network data store 290. In some embodiments, software 250 obtains data from image data store 280, mask data store 285, neural network data store 290, imaging platform 270, and/or user(s) 260 (e.g., by uploading data). In some embodiments, software 250 further includes neural network training module 222 for training one or more neural network models (e.g., stored in neural network data store 290).

In some embodiments, one or more images are obtained from image data store 280. The image data store 280 may be of any suitable type (e.g., database system, multi-file, flat file, etc.) and may store image data in any suitable way and in any suitable format, as aspects of the technology described herein are not limited in this respect. The image data store 280 may be part of or external to computing device 210.

In some embodiments, image data store 280 stores one or more images of tissue from a biological sample, as described herein including at least with respect to FIG. 1A. In some embodiments, the stored image data may have been previously uploaded by a user (e.g., user 260) and/or from one or more public data stores and/or studies. In some embodiments, one or more images stored in the image data store 280 may be processed by the TLS prediction module 212 and/or the TIL prediction module 214. In some embodiments, one or more images stored in the image data store 280 may be used to train one or more neural network models (e.g., with the neural network training module).

In some embodiments, one or more masks are obtained from mask data store 285. The mask data store 285 may be of any suitable type (e.g., database system, multi-file, flat file, etc.) and may store image data in any suitable way and in any suitable format, as aspects of the technology described herein are not limited in this respect. The image data store 280 may be part of or external to computing device 210.

In some embodiments, mask data store 285 stores one or more masks obtained for images of tissue from a biological sample, as described herein including at least with respect to FIG. 1A. In some embodiments, the stored mask data may have been previously uploaded from the TLS prediction module 212, the TIL prediction module 214, by a user (e.g., user 260) and/or from one or more public data stores and/or studies. In some embodiments, one or more masks stored in the mask data store 285 may be processed by the boundary identification module 216.

In some embodiments, the TLS prediction module 212 obtains (either pulls or is provided) a trained neural network model from the neural network data store 290. Additionally, or alternatively, in some embodiments, the TIL prediction module 214 obtained (either pulls or is provided) a trained neural network model from the neural network data store 290. The neural network models may be provided via a communication network (not shown), such as Internet or any other suitable network, as aspects of the technology described herein are not limited to any particular communication network.

In some embodiments, the neural network data store 290 stores one or more neural network models used to identify at least one TLS in an image and/or one or more neural network models used to identify at least one TIL in an image. In some embodiments, the neural network data store 290 includes any suitable data store, such as a flat file, a data store, a multi-file, or data storage of any suitable type, as aspects of the technology described herein are not limited to any particular type of data store. The neural network data store 290 may be part of software 250 (not shown) or excluded from software 250, as shown in FIG. 2. In some embodiments, the neural network data store 290 may store parameter values for trained neural network model(s). When the stored trained NN model(s) are loaded and used, for example by the TLS prediction module 212 and/or the TIL prediction module 214, the parameter values of the trained NN model are loaded and stored/manipulated in memory using at least one data structure.

In some embodiments, the neural network training module 222 may be configured to train one or more neural network models to identify at least one TLS in an image of tissue. Additionally, or alternatively, the neural network training module 222 may be configured to train one or more neural network models to identify at least one TIL in an image of tissue. In some embodiments, the neural network training module 222 trains a neural network model(s) using a training set of image data. For example, the neural network training module 222 may obtain training data from the image data store 280, imaging platform 270, and/or user(s) 260 (e.g., by uploading). In some embodiments, the neural network training module 222 may provide the trained neural network model(s) to the neural network data store 290 for storage therein.

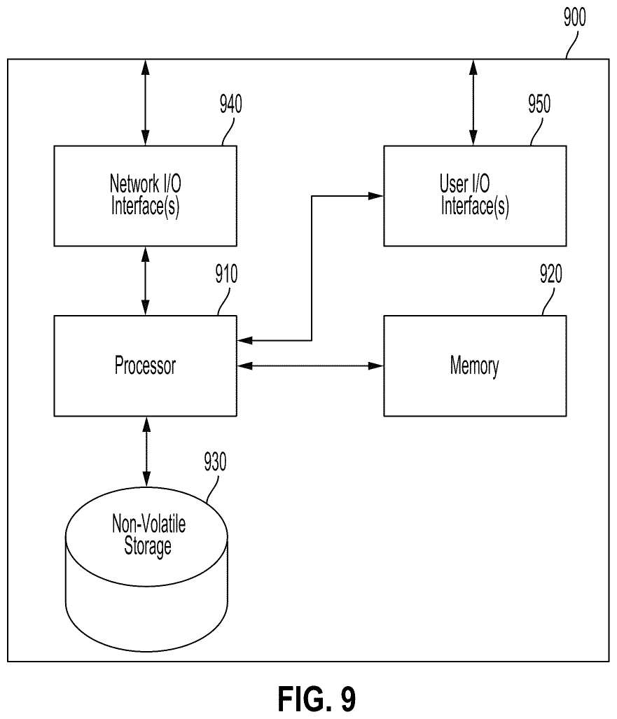

FIG. 3A is a flowchart of an illustrative process 300 for identifying at least one TLS in an image of tissue, according to some embodiments of the technology described herein. One or more acts (e.g., all acts) of process 300 may be performed automatically by any suitable computing device(s). For example, the act(s) may be performed by a laptop computer, a desktop computer, one or more servers, in a cloud computing environment, computing device 900 as described herein including at least with respect to FIG. 9, and/or in any other suitable way.

At act 302, an image of tissue is obtained. In some embodiments, the image of tissue includes an image obtained or previously obtained using an imaging platform such as, for example, imaging platform 104 used to obtain image 106 in FIG. 1A. The image may be a single channel image or a multi-channel image (e.g., a 3-channel RGB image). For example, the image may include a whole slide image (WSI). The image may have any suitable dimensions, as aspects of the technology are not limited in this respect. For example, the image may have at least 100,000×100,000 pixel values per channel, 75,000×75,000 pixel values per channel, 50,000×50,000 pixel values per channel, 25,000×25,000 pixel values per channel, 10,000×10,000 pixel values per channel, 5,000×5,000 pixel values per channel or any other suitable number of pixels per channel. The dimensions of the image may be within any suitable range such as, for example, 50,000-500,000×50,000-500,000 pixel values per channel, 25,000-1 million×25,000-1 million pixel values per channel, 5,000-2 million×5,000-2 million pixel values per channel, or any other suitable range within these ranges.

At (optional) act 304, at least a portion of the image is processed using any suitable pre-processing technique(s). In some embodiments, pre-processing an image includes normalizing the values of pixels in the image. In some embodiments, the normalization is performed elementwise for each channel of the image. In some embodiments, normalizing the values of pixels in an image includes dividing the pixel values by the maximum pixel value in the sub-image such that all pixel values fall within a particular interval (e.g., 0 to 1). For example, for a pixel value that is an unsigned 8-byte integer type (e.g., having a value between 0 to 255), the pixel value may be normalized by dividing the pixel value by 255. Additionally, or alternatively, in some embodiments, normalizing the values of pixels in the image includes performing a Z-score normalization. For example, this may include determining a pre-processed pixel value (P) for each pixel of each channel of the sub-image using Equation 1:

$$P = \frac{\frac{{P}_{input}}{{P}_{max}} - {μ}_{c}}{{σ}_{c}} \qquad ( Equation 1 )$$

Where Pᵢₙₚᵤₜ is the input pixel value, Pₘₐₓ is the maximum pixel value (e.g., 255 for an unsigned 8-byte integer type), μ_{c} is the mean pixel value of a set of reference images for a particular channel, and σ_{c} is the standard deviation of the pixel values of the set of reference images for a particular channel. For example, the mean and standard deviation may include a mean and standard deviation of pixel values in the ImageNet dataset described by Deng, J., et. al. (“ImageNet: A large-scale hierarchical image database.” In 2009 IEEE conference on computer vision and pattern recognition (pp. 248-255)), which is incorporated by reference herein in its entirety. Table 1 lists an example mean and standard deviation per channel for an RGB image:

TABLE 1
Example mean and standard deviation.

| Channel | μ_{c} | σ_{c} |
|---|---|---|
| Red | 0.485 | 0.229 |
| Green | 0.456 | 0.224 |
| Blue | 0.406 | 0.225 |

At act 306, a tertiary lymphoid structure (TLS) mask is obtained for the image of tissue. In some embodiments, the TLS mask indicates, for each particular pixel of multiple pixels of the image, a respective numeric value indicative of the likelihood that the particular pixel is part of the at least one TLS. In some embodiments, a pixel is part of at least one TLS when the pixel is part of a group of one or more pixels in the image that depict at least one TLS (e.g., when a part of the image having the one or more pixels is displayed, the displayed part shows the at least one TLS).

In some embodiments, the TLS mask was previously obtained for the image. For example, the TLS mask may have been previously obtained by a user annotating the image to indicate pixels that are part of at least one TLS. Additionally, or alternatively, the TLS mask may have been previously obtained by processing the image using a neural network model trained to predict, for each particular pixel of multiple pixels in the image, a numeric value indicative of the likelihood that the pixel is part of at least one TLS. In some embodiments, obtaining the TLS mask includes processing the image using such a neural network model. Example techniques for obtaining a TLS mask are described herein including at least with respect to FIG. 3B and in International Publication Number WO2023/154573, which is incorporated by reference herein in its entirety.

At act 308, at least a portion of the image is processed using a trained neural network model to obtain a tumor infiltrating lymphocyte (TIL) mask. In some embodiments, the TIL mask indicates, for each particular pixel of pixels of at least the portion of the image, a respective numeric value indicative of a likelihood that the particular pixel is part of a TIL. In some embodiments, a pixel is part of a TIL when the pixel is part of a group of one or more pixels in the image that depict the TIL (e.g., when a part of the image having the one or more pixels is displayed, the displayed part shows the at least one TIL).

In some embodiments, processing the portion of the image using a trained neural network model to obtain the TIL mask includes: (a) obtaining a set of sub-images of the portion of the image, (b) processing the set of sub-images using the trained neural network model to obtain a respective set of sub-image masks, and (c) generating the TIL mask using the sub-image masks. For example, in some embodiments, the neural network model may be trained to predict, for a particular sub-image, a numeric value indicative of the likelihood that the sub-image includes a TIL and/or a likelihood that the sub-image does not include the TIL. In such an example, the sub-image mask obtained for the sub-image may indicate, for each of at least some of the pixels in the sub-image, the numeric value indicative of the likelihood. Example techniques for obtaining a TIL mask are described herein including at least with respect to FIG. 3C.

At act 310, boundaries of at least one TLS in the image are identified using the TLS mask, the TIL mask, and (optionally) at least one other feature in the image. In some embodiments, this includes: (a) identifying, using the TLS mask, at least one portion (e.g., one or more portions) of the image that the TLS mask indicates is likely to include at least one TLS (e.g., one or more TLSs), (b) identifying, using the TIL mask, at least one portion of the image that the TIL mask indicates is likely to include at least one TIL (e.g., one or more TILs), (c) (optionally) identifying at least a portion of the image that likely includes the feature(s), and (d) identifying overlaps between the portion(s) of the image likely to include the TLS(s), portion(s) of the image likely to include the TIL(s), and (optionally) the portion(s) of the image likely to include the feature(s). It should be appreciated that acts (a), (b), and (c) may be performed in any suitable order, as aspects of the technology described herein are not limited in this respect.

In some embodiments, as described herein, the TLS mask may indicate, for each of multiple pixels in the image, a numeric value indicative of the likelihood that the pixel is part of a TLS. In some embodiments, to assist in identifying portion(s) of the image likely to include TLS(s), the TLS mask is first processed to generate a binary version of the TLS mask. Generating the binary version of the TLS mask may include comparing each of at least some of the values of the TLS mask to a threshold value (e.g., a threshold value that is determined in advance of performance of process 300 or dynamically determined as part of process 300) and setting each pixel value to 0 or 1 depending on the result of the comparison. For example, when the value of the TLS mask is greater than or equal to the threshold value, then the value may be set to 1 and when the value of the TLS mask does not exceed the threshold value, then the value may be adjusted to 0, or vice versa. The threshold value may be any suitable threshold value such as aspects of the technology described herein are not limited in this respect. For example, the threshold may be at least 25%, 40%, 50%, 60%, or 75% of the maximum value in the TLS mask. For example, when the TLS mask includes values between 0 and 1, the threshold may be at least 0.25, at least 0.40, at least 0.50, at least 0.60, at least 0.75, or any other suitable threshold value.

In some embodiments, the binary version of the TLS mask is used to identify portion of the image that likely include at least one TLS. For example, one of the binary values (e.g., 0 or 1) of the TLS mask may indicate pixels likely to be part of at least one TLS. Identifying the portions of the image likely to include at least one TLS may include identifying the pixels which the mask indicates are likely to include at least one TLS. Additionally, or alternatively, a border-following algorithm may be applied to the mask and used to identify the portion(s) of the image likely to include at least one TLS. For example, pixels positioned within the identified boundaries may be identified as being part of at least one TLS. As a nonlimiting example, the borders may be identified using the border-following algorithm described by S. Suzuki, et. al. (“Topological structural analysis of digitized binary images by border following”. Computer Vision, Graphics, and Image Processing, 30(1):32-46, 1985), which is incorporated by reference herein in its entirety. In some embodiments, one or more parameters of the border-following algorithm may be set to identify the boundaries of the at least one TLS. For example, the parameters may include a parameter to select a contour approximation method (e.g., method=CHAIN_APPROX_SIMPLE). As another example, the parameters may include a parameter for identifying outer contours from among nested contours (e.g., hierarchy=RETR_EXTERNAL).

In some embodiments, as described herein, the TIL mask may indicate, for each of multiple pixels in the image, a numeric value indicative of the likelihood that the pixel is part of a TIL. In some embodiments, to assist in identifying portion(s) of the image likely to include TIL(s), the TIL mask is first processed to generate a binary version of the TIL mask. Generating the binary version of the TIL mask may include comparing each of at least some of the values of the TIL mask to a threshold value (e.g., a threshold value that is determined in advance of performance of process 300 or dynamically determined as part of process 300) and setting each pixel value to 0 or 1 depending on the result of the comparison. For example, when the value of the TIL mask is greater than or equal to the threshold value, then the value may be set to 1 and when the value of the TIL mask does not exceed the threshold value, then the value may be adjusted to 0, or vice versa. The threshold value may be any suitable threshold value such as aspects of the technology described herein are not limited in this respect. For example, the threshold may be at least 25%, 40%, 50%, 60%, or 75% of the maximum value in the TIL mask. For example, when the TIL mask includes values between 0 and 1, the threshold may be at least 0.25, at least 0.40, at least 0.50, at least 0.60, at least 0.75, or any other suitable threshold value.

In some embodiments, the binary version of the TIL mask is used to identify portion(s) of the image that likely include at least one TIL. For example, one of the binary values (e.g., 0 or 1) of the TIL mask may indicate pixels likely to be part of at least one TIL. Identifying the portions of the image likely to include at least one TIL may include identifying the pixels which the mask indicates are likely to include at least one TIL.

In some embodiments, the at least one other feature of the image may include any suitable feature such as tissue, tumor tissue, and/or non-tumor tissue.

In some embodiments, the feature of tissue may refer to portions of the image that include tissue, as opposed to background (e.g., the container holding the biological sample). Additionally, or alternatively, the feature of tissue may refer to portions of the image that include tissue and exclude image artifacts. In some embodiments, tissue in the image may be identified using any suitable techniques, as aspects of the technology described herein are not limited in this respect. For example, a user may manually identify and/or annotate regions of the image that include tissue. Additionally, or alternatively, the image may be processed (e.g., using image segmentation techniques) to identify the regions of the image that include tissue. In some embodiments, identifying the boundaries of the at least one TLS using the TLS mask, the TIL mask, and the feature of tissue includes identifying portions of the image that include all three features: TLS(s), TIL(s), and tissue, as opposed to portion(s) of the image that include image artifacts and/or background. In some embodiments, identifying the portions of the image that include TLS(s), TIL(s), and tissue includes identifying overlap between portions of the image identified as likely including TLS(s), portions of the image identified as likely including TIL(s), and portions of the image identified as likely including tissue.

In some embodiments, the feature of tumor tissue may refer to portions of the image that include tissue having at least a threshold amount, number, or proportion of tumor cells (e.g., as opposed to non-tumor cells). In some embodiments, tumor tissue may be identified using any suitable techniques, as aspects of the technology described herein are not limited in this respect. For example, a user may manually identify the portions of the image that include tumor tissue. Additionally, or alternatively, the image may be processed (e.g., using image segmentation techniques) to identify the portions of the image that include the tumor tissue. In some embodiments, identifying the boundaries of the at least one TLS mask, TIL mask, and feature of tumor issue includes identifying portions of the image that include all three features: TLS(s), TIL(s), and tumor tissue. In some embodiments, identifying the portions of the image that include TLS(s), TIL(s), and tumor tissue includes identifying overlap between portions of the image identified as likely including TLS(s), portions of the image identified as likely including TIL(s), and portions of the image identified as likely including tumor tissue.

In some embodiments, the feature of non-tumor tissue may refer to portions of the image that include tissue having less than the threshold amount, number, or proportion of tumor cells. In some embodiments, non-tumor tissue may be identified using any suitable techniques, as aspects of the technology described herein are not limited in this respect. For example, a user may manually identify the portions of the image that include non-tumor tissue. Additionally, or alternatively, the image may be processed (e.g., using image segmentation techniques) to identify the portions of the image that include the non-tumor tissue. In some embodiments, identifying the boundaries of the at least one TLS mask, TIL mask, and feature of non-tumor tissue includes identifying portions of the image that include all three features: TLS(s), TIL(s), and non-tumor tissue. In some embodiments, identifying the portions of the image that include TLS(s), TIL(s), and non-tumor tissue includes identifying overlap between portions of the image identified as likely including TLS(s), portions of the image identified as likely including TIL(s), and portions of the image identified as likely including tumor tissue.

At (optional) act 312, a filter may be applied to the identified boundaries of the at least one TLS. In some embodiments, applying the filter to a particular set of boundaries includes determining whether an area of the region enclosed by the boundaries is greater than or equal to a threshold, and filtering out the boundaries (e.g., excluding from further processing) when the area is not greater than or equal to the threshold. The threshold may include any suitable threshold, as aspects of the technology described herein are not limited in this respect. For example, the threshold may be at least 100 pixels, at least 500 pixels, at least 1,000 pixels, at least 1,500 pixels, at least 2,000 pixels, at least 2,500 pixels, at least 3,000 pixels, at least 3,500 pixels, at least 4,000 pixels, at least 4,500 pixels, at least 5,000 pixels, at least 5,500 pixels, at least 6,000 pixels, at least 6,500 pixels, at least 7,000 pixels, at least 7,500 pixels, at least 8,000 pixels, at least 9,000 pixels, at least 10,000 pixels, or any other suitable threshold. Additionally, or alternatively, the threshold may be a value between 100 and 10,000 pixels, 500 and 9,000 pixels, 1,000 and 8,000 pixels, 2,000 and 7,000 pixels, 3,000 and 6,000 pixels, 4,000 and 5,000 pixels, or within any other suitable range of pixels.

At act 314, one or more characteristics of the at least one TLS are identified using the boundaries of the at least one TLS in the image.

The characteristics may include any suitable characteristic that may be obtained using one or more of the boundaries identified at act 310. As a nonlimiting example, the characteristics may include the number of TLSs in at least a portion of the image. This may be determined by counting the number of bounded regions (e.g., regions enclosed by the identified boundaries) in the portion of the image.

Additionally, or alternatively, the characteristics may include the number of TLSs in at least a portion of an image normalized by the area of the portion of the image (which may be termed “TLS density”). In some embodiments, this characteristic may be determined by dividing the number of TLSs in the portion of the image by the total area of that portion of the image. The area of the portion of the image may include both the area(s) of the portion of the image that include TLSs (e.g., bounded regions) and the area(s) of the portion of the image that do not include TLSs (e.g., non-bounded regions). In some embodiments, the area Aᵢ of a portion i of an image may be determined using Equation 2:

$${A}_{i} = Pixel Size * Pixel Area \qquad ( Equation 2 )$$

where:

$$Pixel Size = Pixel Height * Pixel Width \qquad ( Equation 3 )$$

$$Pixel Area = {i}_{height} * {i}_{width} \qquad ( Equation 4 )$$

where i_{height} is the height of the portion i of the image as measured in number of pixels and i_{width} is the width of the portion i of the image as measured in number of pixels. In some embodiments, pixel height and pixel width may be measured in millimeter per pixel. Pixel height and pixel width may be obtained from annotation information associated with the image. For example, when the image is a WSI, the pixel height and width may be obtained from the WSI file. In some embodiments, if the pixel height and width information is not available, then default values for pixel height and pixel width may be used. For example, the default value for pixel height and width may include a value of at least 0.4 μm per pixel, 0.5 μm per pixel, 0.6 μm per pixel, 0.7 μm per pixel, 0.8 μm per pixel, between 0.1 μm per pixel and 2 μm per pixel, between 0.2 μm per pixel and 1 μm per pixel, or any other suitable value.

Additionally, or alternatively, the characteristics identified at act 314 may include the total area of TLSs in at least a portion of the image. In some embodiments, determining the total area of TLSs includes determining the area of each region of the portion of the image that is enclosed by the boundaries identified at act 310. In some embodiments, the area of a bounded region may be determined using any suitable technique(s), as aspects of the technology described herein are not limited in this respect. For example, the area of a bounded region may be determined by the shoelace algorithm described in Braden, B. (“The Surveyor's Area Formula.” In The College Mathematics Journal, (1996), Volume 17, Number 4, pp. 326-337). As another example, the Shapely Python package (Gillies, S., et. al. “Shapely: manipulation and analysis of geometric objects.” 2007. Available from: https://github.com/Toblerity/Shapely.) may be used to determine the area of the bounded region. In particular, the area method of “shapely.geometry.Polygon” class and/or the area method of “shapely.geometry.MultiPolygon” class may be used to determine the area of the bounded region.

Additionally, or alternatively, the characteristics identified at act 314 may include the total area of TLSs in at least a portion of the image normalized by the area of the portion of the image. This may be determined, in some embodiments, by dividing the total area of TLSs by the area of the portion of the image. Techniques for determining the total area of the TLSs and for determining the area of a portion of an image are described herein.

Additionally, or alternatively, the characteristics identified at act 314 may include the median area of TLSs in at least a portion of the image. In some embodiments, the median TLS area is determined by determining the area of each bounded region (e.g., enclosed by the boundaries identified at act 314) in the portion of the image. For example, the area of each bounded region may be determined using the techniques described above for determining the area of a bounded region. In some embodiments, the median of the determined areas is identified as the median TLS area in the portion of the image.

Additionally, or alternatively, the characteristics identified at act 314 may include the median area of TLSs in at least a portion of the image normalized by the area of the portion of the image. The area of the portion of the image may include both the areas of the portion of the image that include TLSs (e.g., bounded regions) and the areas of the portion of the image that do not include TLSs (e.g., non-bounded regions). In some embodiments, the median area of TLSs and the area of the portion of the image are determined using the techniques described herein.

Additionally, or alternatively, the characteristics identified at act 314 may include characteristics of at least one TLS associated with a particular feature (e.g., tumor tissue, non-tumor tissue, and/or tissue). A TLS may be associated with a particular feature when its boundaries were identified using the feature at act 310. Identifying characteristics of at least one TLS associated with a particular feature may include identifying characteristics of at least one TLS associated with tissue. Additionally, or alternatively, identifying characteristics of at least one TLS associated with a particular feature may include identifying characteristics of at least one TLS associated with tumor tissue. Additionally, or alternatively, identifying characteristics of at least one TLS associated with a particular feature may include identifying characteristics of at least one TLS associated with non-tumor tissue. In some embodiments, identifying characteristics of at least one TLS associated with non-tumor tissue includes identifying characteristics of at least one TLS associated with borderline tumorous tissue and/or identifying characteristics of at least one TLS associated with non-borderline tumorous tissue. Borderline tumorous tissue may include non-tumor tissue that is within a threshold distance of tumor tissue. Non-borderline tumorous tissue may include non-tumor tissue that is positioned at a distance greater than or equal to the threshold distance with respect to the tumor tissue.

In some embodiments, by identifying characteristics of TLS(s) associated with different features, it is possible to obtain a more comprehensive understanding of the subject's immune response. For example, it may be useful to compare the number and/or area of TLSs in non-tumor tissue, borderline tumorous tissue, and tumor tissue to evaluate the subject's immune response.

At (optional) act 316, one or more characteristics of at least one TIL in the image may be identified using the TIL mask. For example, as described herein with respect to act 310, a binary version of the TIL mask may be generated and used to identify portion(s) of the image that likely include at least one TIL. Identifying the one or more characteristics of the at least one TIL may include identifying characteristics of the portion(s) of the image identified as likely including at least one TIL. In some embodiments, identifying the characteristics of the at least one TIL includes determining an area of the at least one TIL. The area may be determined based on the sum of pixels corresponding to the at least one TIL. The sum of the pixels may be converted to area using Equation 2, Equation 3, and Equation 4. Additionally, or alternatively, in some embodiments, identifying the one or more characteristics of the at least one TIL may include determining an area of the at least one TIL in a portion of the image normalized by the area of the portion of the image.

Additionally, or alternatively, the characteristics identified at (optional) act 316 may include characteristics of at least one TIL associated with a particular feature (e.g., tumor tissue, non-tumor tissue, and/or tissue). A TIL may be associated with a particular feature when its boundaries are determined based on an overlap with the feature (e.g., where the TIL indicated by the TIL mask overlaps tissue, tumor tissue, non-tumor tissue, etc.). Identifying characteristics of at least one TIL associated with a particular feature may include identifying characteristics of at least one TIL associated with tissue. Additionally, or alternatively, identifying characteristics of at least one TIL associated with a particular feature may include identifying characteristics of at least one TIL associated with tumor tissue. Additionally, or alternatively, identifying characteristics of at least one TIL associated with a particular feature may include identifying characteristics of at least one TIL associated with non-tumor tissue. In some embodiments, identifying characteristics of at least one TIL associated with non-tumor tissue includes identifying characteristics of at least one TIL associated with borderline tumorous tissue and/or identifying characteristics of at least one TIL associated with non-borderline tumorous tissue. Borderline tumorous tissue may include non-tumor tissue that is within a threshold distance of tumor tissue. Non-borderline tumorous tissue may include non-tumor tissue that is positioned at a distance greater than or equal to the threshold distance with respect to the tumor tissue.

As described herein, one or more of TLS and/or TIL characteristics may be used as prognostic or predictive biomarkers for diagnosing the subject, predicting overall survival for the subject, and/or predicting how the subject will respond to a particular therapy. At (optional) act 318, a treatment is identified for the subject based on at least one of the characteristics identified at act 314 and/or (optional) act 316. In some embodiments, this may include determining whether the characteristic satisfies at least one criterion and identifying a treatment for the subject based on an evaluation of whether the characteristics satisfy the at least one criterion. For example, this may include determining whether the at least one characteristic exceeds a particular threshold and identifying a treatment for the subject based on the result of comparing the at least one characteristic to the threshold.

As a nonlimiting example, the number of TLSs in at least a portion of an image normalized by the area of the portion of the image (“TLS density”) may be used to determine whether to recommend administering an immunotherapy to the subject. The TLS density may be compared to a threshold density, and when the TLS density exceeds the threshold density, then an immunotherapy may be recommended for administering to the subject. The immunotherapy may include any suitable immunotherapy including, for example, at least one of the immunotherapies described in the “Methods of Treatment” section. In some embodiments, the threshold density includes any suitable threshold such as, for example, at least 0.25 TLS/mm², at least 0.5 TLS/mm², at least 0.75 TLS/mm², at least 0.8 TLS/mm², at least 0.9 TLS/mm², at least 0.95 TLS/mm², at least 1.0 TLS/mm², at least 1.25 TLS/mm², at least 1.5 TLS/mm², at least 1.75 TLS/mm², at least 1.8 TLS/mm², at least 1.9 TLS/mm², at least 2.0 TLS/mm², at least 2.1 TLS/mm², at least 2.25 TLS/mm², at least 2.5 TLS/mm², at least 2.75 TLS/mm², at least 3.0 TLS/mm², within the range of 0-15 TLS/mm², 0-10 TLS/mm², 1-5 TLS/mm², or any other suitable threshold, as aspects of the technology are not limited in this respect. Consider, for example, a subject with basal-like breast cancer. In some embodiments, the techniques described herein may be used to determine a TLS density for the subject. If the TLS density exceeds a threshold density such as, for example, a threshold TLS density of 2 TLS/mm², then an immunotherapy may be identified for the subject. Consider, as another example, a subject with lung adenocarcinoma. In some embodiments, the techniques described herein may be used to determine the TLS density for the subject. If the TLS density exceeds a threshold density such as, for example, a threshold TLS density of 1.22 TLS/mm², then an immunotherapy may be identified for the subject.

In some embodiments, an output is generated. In some embodiments, the output may be stored (e.g., in a non-transitory memory), displayed via a user interface, transmitted to one or more other devices, or otherwise processed using any suitable techniques, as aspects of the technology are not limited in this respect. For example, the output may be displayed using a graphical user interface (GUI) of a computing device. As another example, the output may be included as part of an electronically-generated report. In some embodiments, the output may include any suitable output such as, for example, at least a portion of the image of tissue, the determined TLS mask, the determined TIL mask, one or more of the TLS sub-image masks, a mask of at least one other feature (e.g., tissue, tumor tissue, and/or non-tumor tissue), one or more TIL masks, any identified TLS boundaries, any identified TIL boundaries, boundaries of at least one other feature, any identified TLS characteristics, any identified TIL characteristics and/or any treatment(s) recommended for the subject.

At (optional) act 320, the treatment identified at act 318 is administered to the subject. Techniques for administering the treatment are described herein including at least in the “Methods of Treatment” section.

FIG. 3B is a flowchart of an illustrative process 330 for generating a TLS mask for an image of tissue, according to some embodiments of the technology described herein. One or more acts (e.g., all acts) of process 330 may be performed automatically by any suitable computing device(s). For example, the act(s) may be performed by a laptop computer, a desktop computer, one or more servers, in a cloud computing environment, computing device 900 as described herein including at least with respect to FIG. 9, and/or in any other suitable way.

At act 332, a set of overlapping sub-images of an image of tissue is obtained. For example, sub-images of the image obtained at act 302 of FIG. 3A may be obtained. In some embodiments, each sub-image in the set of overlapping sub-images may be obtained, from the image of tissue, in any suitable manner, as aspects of the technology described herein are not limited in this respect. For example, a sub-image may be cropped out of the image of tissue. The dimensions of the sub-image may depend on the dimensions of the image from which the sub-image is obtained. For example, dimensions of the sub-image may be smaller than the corresponding dimensions of the image from which the sub-image is obtained. For example, the sub-image may have at least 128×128 pixels per channel, 256×256 pixels per channel, 512 pixels×512 pixels per channel, 1024×1024 pixels per channel, 2048×2048 pixels per channel, 40964×4096 pixels per channel, 8192×8192 pixels per channel or any other suitable number of pixels per channel. The dimensions of the sub-image image may be within any suitable range such as, for example, 10-100,000×10-100,000 pixel values per channel, 100-50,000×100-50,000 pixel values per channel, 1,000-10,000×1,000-10,000 pixel values per channel, or any other suitable range within these ranges.

In some embodiments, sub-images in the set of sub-images overlap one another. For example, a pair of sub-images that overlap one another may each include the same subset of pixels corresponding to the image from which the sub-images were obtained. The sub-images may overlap one another in any suitable direction, as aspects of the technology are not limited in this respect. For example, sub-images in the set of overlapping sub-images may overlap one another along a horizontal and/or along a vertical axis. In some embodiments, sub-images in the set of overlapping sub-images overlap one another by any suitable degree of overlap, as aspects of the technology are not limited in this respect. For example, in a particular direction, the sub-images may overlap one another by at least 90%, at least 80%, at least 75%, at least 60%, at least 50%, at least 40%, at least 35%, at least 25%, at least 20%, at least 10%, between 10% and 90%, or between 40% and 60%. For example, sub-images having dimensions of 512 pixels per channel×512 pixels per channel may overlap one another by 256 pixels in both the horizontal and vertical direction.

In some embodiments, the set of overlapping sub-images includes any suitable number of sub-images, as aspects of the technology are not limited in this respect. For example, the set of overlapping sub-images may include at least 10, at least 50, at least 75, at least 100, at least 150, at least 250, at least 300, at least 500, at least 750, at least 1000, at least 2,500, at least 5,000, at least 7,500, at least 10,000, at least 25,000, between 10 and 25,000, between 100 and 10,000 sub-images, or any other suitable number of sub-images. In some embodiments, each sub-image in the set of overlapping sub-images overlaps at least one another sub-image in the set of overlapping sub-images.

In some embodiments, the set of overlapping sub-images cover at least a portion of the image of tissue. For example, when the image of tissue is a WSI, the set of overlapping sub-images may cover at least a portion of the WSI. The portion of the image covered by the set of overlapping sub-images may include any suitable portion such as, for example, at least 5% of the image, at least 10%, at least 25%, at least 50%, at least 60%, at least 75%, at least 80%, at least 90%, at least 95%, at least 98%, 100%, between 5% and 100%, between 25% and 80%, or any other suitable portion, as aspects of the technology are not limited in this respect.

At act 334, the set of overlapping sub-images is processed using a TLS neural network model to obtain a respective set of pixel-level sub-image masks. In some embodiments, a sub-image mask indicates, for each particular pixel in a respective sub-image, a respective numeric value indicative of a likelihood that the particular pixel is part of a TLS.

In some embodiments, processing the overlapping sub-images using the TLS neural network mask includes processing a first sub-image using the trained neural network model to obtain a respective first pixel-level sub-image mask, processing a second sub-image using the same trained neural network model to obtain a respective second pixel-level sub-image mask, processing a third sub-image using the same trained neural network model to obtain a third respective pixel-level sub-image mask, and so on. In some embodiments, processing the set of overlapping images includes processing some or all of the sub-images in the set of overlapping sub-images using the trained neural network model. In some embodiments, the trained neural network may be any suitable semantic segmentation deep neural network. A semantic segmentation deep neural network may be any neural network that identifies labels for individual pixels (e.g., some or all pixels in an image). In some embodiments, the neural network may have any of the example architectures described in International Publication Number WO2023/154573, which is incorporated by reference herein in its entirety.

In some embodiments, a pixel-level sub-image mask indicates, for each of multiple pixels in the pixel-level sub-image mask, a respective probability that the particular pixel is part of a TLS. For example, a first pixel-level sub-image mask for a first sub-image may indicate a probability that a first pixel in a first sub-image is part of a TLS.

As described herein, in some embodiments, sub-images in the set of overlapping sub-images overlap one another. Sub-images that overlap one another may share pixels that are in an overlapping region of the sub-images. Accordingly, in some embodiments, the pixel-level sub-image masks obtained for overlapping sub-images may each, for the same pixel, indicate a probability that the pixel is part of a TLS. Because different sub-images include different information (e.g., values of pixels that are not included in the overlapping region) that is processed using the trained neural network model, the multiple probabilities predicted for the pixel using the neural network model may differ from one another. Accordingly, in some embodiments, process 300 includes techniques for accounting for the multiple different predicted probabilities. For example, in some embodiments, act 336 of process 330 may be implemented to account for the multiple predictions.

At act 336, a TLS mask is generated for the image using the set of pixel-level sub-image masks. In some embodiments, this includes using at least some of the set of pixel-level sub-image masks corresponding to the at least some of the set of overlapping sub-images covering at least the portion of the image. For example, the pixel-level sub-image masks may be used to determine for each of multiple pixels in a region of overlap between two or more overlapping sub-images, an average probability that the pixel is part of TLS. In some embodiments, determining the average includes determining a weighted average. For example, weighting may be performed such that values in the pixel-level sub-image mask that are positioned closer to the center of the sub-image mask are made to contribute more to the average than the values in the pixel-level sub-image mask that are positioned closer to the borders of the sub-image mask. This helps to reduce artifacts/errors at image edges and leads to overall improved performance in accurately identifying TLS structures.

FIG. 3C is a flowchart of an illustration process 340 for generating a tumor infiltrating lymphocyte (TIL) mask for an image of tissue, according to some embodiments of the technology described herein. One or more acts (e.g., all acts) of process 340 may be performed automatically by any suitable computing device(s). For example, the act(s) may be performed by a laptop computer, a desktop computer, one or more servers, in a cloud computing environment, computing device 900 as described herein including at least with respect to FIG. 9, and/or in any other suitable way.

At act 342, a set of sub-images of an image of tissue is obtained. In some embodiments, each sub-image in the set of sub-images may be obtained, from the image of tissue, in any suitable manner, as aspects of the technology described herein are not limited in this respect. For example, a sub-image may be cropped out of the image of tissue. The dimensions of the sub-image may depend on the dimensions of the image from which the sub-image is obtained. For example, dimensions of the sub-image may be smaller than the corresponding dimensions of the image from which the sub-image is obtained. For example, the sub-image may have at least 128×128 pixels per channel, 256×256 pixels per channel, 512 pixels×512 pixels per channel, 1024×1024 pixels per channel, 2048×2048 pixels per channel, 40964×4096 pixels per channel, 8192×8192 pixels per channel or any other suitable number of pixels per channel. The dimensions of the sub-image image may be within any suitable range such as, for example, 10-100,000×10-100,000 pixel values per channel, 100-50,000×100-50,000 pixel values per channel, 1,000-10,000×1,000-10,000 pixel values per channel, or any other suitable range within these ranges.

In some embodiments, the set of sub-images includes any suitable number of sub-images, as aspects of the technology are not limited in this respect. For example, the set of sub-images may include at least 10, at least 50, at least 75, at least 100, at least 150, at least 250, at least 300, at least 500, at least 750, at least 1000, at least 2,500, at least 5,000, at least 7,500, at least 10,000, at least 25,000, between 10 and 25,000, between 100 and 10,000 sub-images, or any other suitable number of sub-images. In some embodiments, each sub-image in the set of sub-images overlaps at least one another sub-image in the set of sub-images.

In some embodiments, the set of sub-images cover at least a portion of the image of tissue. For example, when the image of tissue is a WSI, the set of sub-images may cover at least a portion of the WSI. The portion of the image covered by the set of sub-images may include any suitable portion such as, for example, at least 5% of the image, at least 10%, at least 25%, at least 50%, at least 60%, at least 75%, at least 80%, at least 90%, at least 95%, at least 98%, 100%, between 5% and 100%, between 25% and 80%, or any other suitable portion, as aspects of the technology are not limited in this respect.

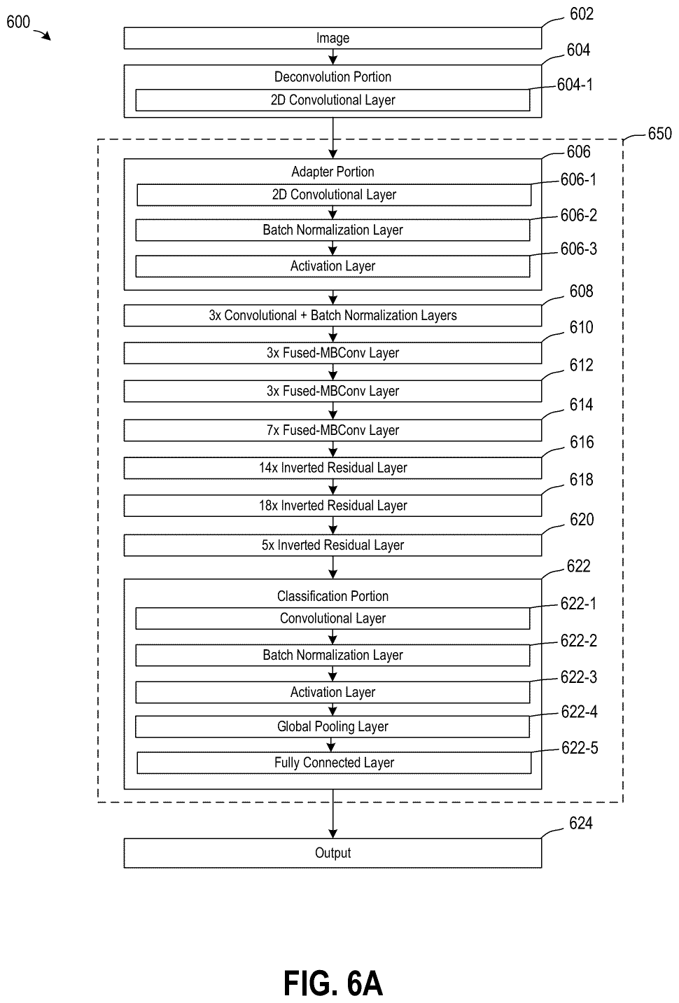

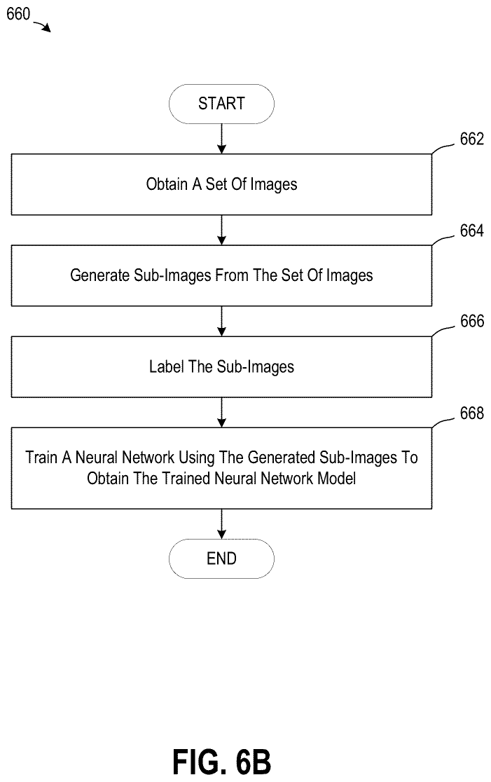

At act 344, the set of sub-images is processed using a TIL neural network to obtain a respective set of sub-image masks. In some embodiments, a sub-image mask indicates, for a respective sub-image, a respective numeric value indicative of a likelihood that pixels in the respective sub-image are part of a TIL. In some embodiments, processing the set of sub-images using the TIL neural network may include processing a first sub-image using the trained neural network model to obtain a respective first sub-image mask, processing a second sub-image using the same trained neural network model to obtain a respective second sub-image mask, processing a third sub-image using the same trained neural network model to obtain a third respective sub-image mask, and so on. In some embodiments, processing the set of sub-images includes processing some or all of the sub-images in the set of sub-images using the trained neural network model. In some embodiments, the trained neural network may be any suitable convolutional neural network (CNN) that classifies images. Nonlimiting examples of CNNs include EfficientNet, EfficientNetV2, ResNet, VGGNet, DenseNet, GoogleNet, or any other suitable type of CNN. In some embodiments, the neural network may have any of the example architectures described herein including at least with respect to the “Neural Network Model” section. In some embodiments, the neural network is trained using and of the neural network training techniques described herein including at least with respect to FIG. 6B.

In some embodiments, the TIL neural network is trained to predict a numeric value indicative of the likelihood that a particular sub-image includes pixels that are part of at least one TIL. Additionally, or alternatively, the TIL neural network may be trained to predict a numeric value indicative of the likelihood that the particular sub-image does not include pixels that are part of at least one TIL. For example, the output of the TIL neural network may include both a numeric value indicative of the likelihood that the sub-image includes pixels that are part of at least one TIL and a numeric value indicative of the likelihood that the sub-image does not include pixels that are part of the TIL. In this example, the sub-image mask predicted for a particular sub-image may have the dimensions of the input image and may have 2 channels, each of which corresponds to a respective numeric value. As one non-limiting example, a sub-image mask may be obtained for a sub-image having dimensions 128×128 pixels. The first channel of the sub-image mask may be a 128×128 matrix with repeated entries indicating the numeric value indicative of the likelihood that the sub-image mask includes pixels that are part of at least one TIL. The second channel of the sub-image mask may be a 128×128 matrix with repeated entries indicating the numeric value indicative of the likelihood that the sub-image mask does not include pixels that are part of at least one TIL.

In some embodiments, the sub-image mask is post-processed to obtain a sub-image mask having only one channel. In some embodiments, this includes determining the single-channel sub-image mask using the numeric value in the first channel and the numeric value in the second channel. In some embodiments, this includes comparing the numeric value in the first channel to the numeric value in the second channel (and/or other channels). If the numeric value in the first channel is greater than the numeric value in the second channel, then pixels in the single channel are assigned a first value (e.g., 1). If the numeric value in the second channel is greater than the numeric value in the first channel, then pixels in the single channel are assigned a second value (e.g., 0). As a result, the sub-image mask may indicate whether or not the sub-image includes pixels that are part of at least one TIL. For example, a sub-image mask that includes pixels having numeric values of 1 may indicate that pixels in the corresponding sub-image are part of at least one TIL. A sub-image mask that includes pixels having numeric values of 0 may indicate that pixels in the corresponding sub-image are not part of the at least one TIL.

At act 346, a TIL mask is generated for the image using the set of sub-image masks. In some embodiments, this includes using at least some of the set of sub-image masks corresponding to the at least some of the set of sub-images covering at least the portion of the image. In some embodiments, one or more processing techniques may be applied to the sub-image masks to combine sub-image mask data for adjacent or close pixels. For example, predictor window summation may be applied to the sub-image masks to obtain the TIL mask.

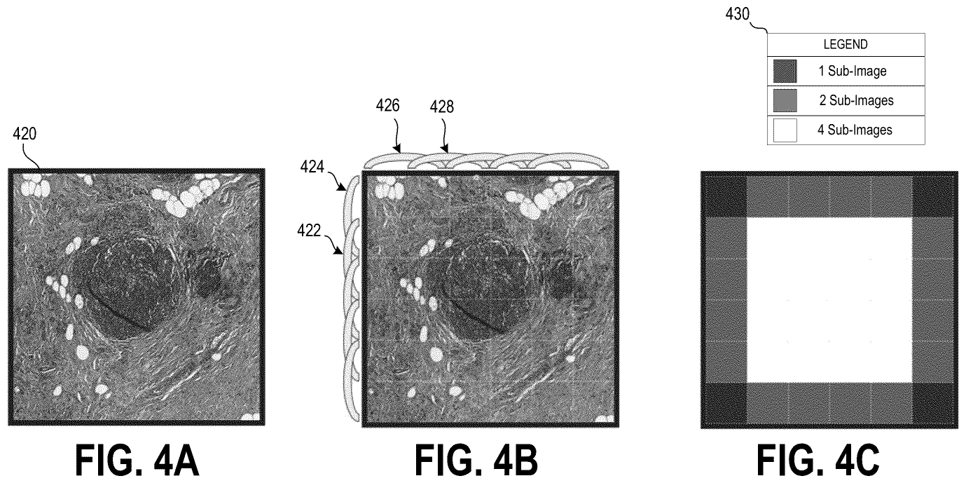

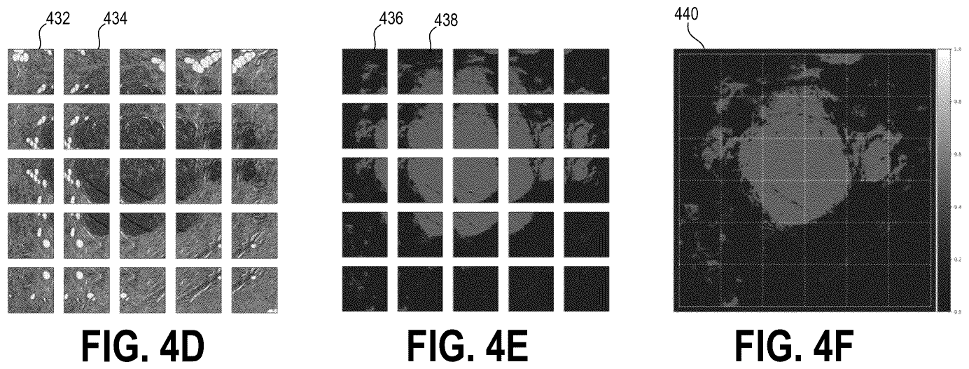

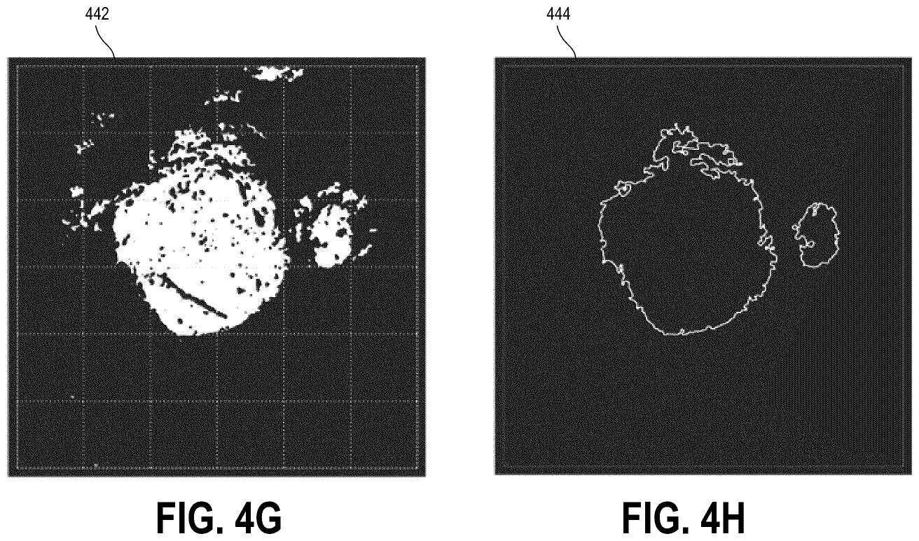

FIGS. 4A, 4B, 4C, 4D, 4E, 4F, 4G, and 4H show an example of using a trained neural network model to identify at least one TLS in an image of tissue, according to some embodiments of the technology described herein.

FIG. 4A shows an example image 420 of tissue obtained from a subject. In some embodiments, image 420 is obtained using any suitable techniques such as, for example, the techniques described herein including at least with respect to FIGS. 1A and 3A.

FIG. 4B shows an example of obtaining sub-images from the image 420. In the example, the dimensions of a first sub-image are indicated by the bracket 426 and bracket 424. The dimensions of a second sub-image are indicated by the bracket 428 and bracket 424. The dimensions of a third sub-image are indicated by the bracket 422 and bracket 426.

In some embodiments, the sub-images are obtained using the techniques described herein, including at least with respect to act 332 of FIG. 3B. For example, the sub-images may be obtained by obtaining crops from the image 420. While the example shows that sub-images are obtained for the entire image 420, it should be appreciated that sub-images may be obtained for only a portion of the image 420.

In the example shown in FIG. 4B, each of the sub-images overlaps at least one other sub-image. For example, the first sub-image overlaps the second sub-image in the horizontal direction, as indicated by the overlapping brackets 426 and 428. The first sub-image also overlaps the third sub-image in the vertical direction, as indicated by the brackets 424 and 422. The second sub-image also overlaps the third sub-image (e.g., the lower left quadrant of the second sub-image is the same as the top right quadrant of the third sub-image).

FIG. 4C is an example showing an indication of the degree of overlap between the sub-images. As indicated by legend 430, different portions of the image are covered by a different number of sub-images. Accordingly, as described herein, including at least with respect to FIG. 3B, a different number of sub-image masks may be generated for the different portions of the image. For example, a single sub-image mask may be generated for portions of the image that are covered by a single sub-image, two sub-image masks may be generated for portions of the image that are covered by two sub-images, and four sub-image masks may be generated for portions of the image that are covered by four sub-image masks.

FIG. 4D shows example sub-images obtained from the image 420 in FIG. 4A. Sub-image 432 corresponds to the sub-image indicated by brackets 424 and 426 in FIG. 4B. Sub-image 434 corresponds to the sub-image indicated by brackets 424 and 428 in FIG. 4B. Because sub-images 432 and 434 correspond to overlapping regions of image 420, sub-images 432 and 434 each share at least some of the same pixels.

FIG. 4E shows example sub-image masks obtained for the sub-images shown in FIG. 4D. For example, the sub-image masks shown in FIG. 4E may be obtained using the machine learning techniques described herein including at least with respect to act 434 of process 340 shown in FIG. 3B.

Because, in this example, the sub-images correspond to overlapping regions of image 420 in FIG. 4A, the determined sub-image masks may include multiple sub-image masks for a same region of pixels. For example, the sub-image masks 436 and 438 generated for sub-images 432 and 434 each include a sub-image mask for the shared region of pixels of the sub-images 432 and 434.

Accordingly, in some embodiments, the example sub-image masks shown in FIG. 4E may be used to determine the single pixel-level mask 440, shown in FIG. 4F, for the image 420. For example, the mask may be determined using the techniques described herein including at least with respect to act 336 of process 330 shown in FIG. 3B.

FIGS. 4G and 4H show an example of processing the determined mask to identify one or more features for at least one TLS in the image 420. Mask 442 is an example of a binarized version of mask 440 in FIG. 4F. For example, as described herein, binarizing the mask may include comparing the probability determined to a particular pixel to a threshold probability and binarizing the mask based on the result of the comparison.

As shown in the example, the mask may be used to identify boundaries 444 of at least one TLS, and the identified boundaries may be used to determine one or more characteristics. Examples of identifying boundaries are described herein including at least with respect to act 310 of process 300 shown in FIG. 3A. Examples of determining characteristics are described herein including at least with respect to act 314 of process 300.

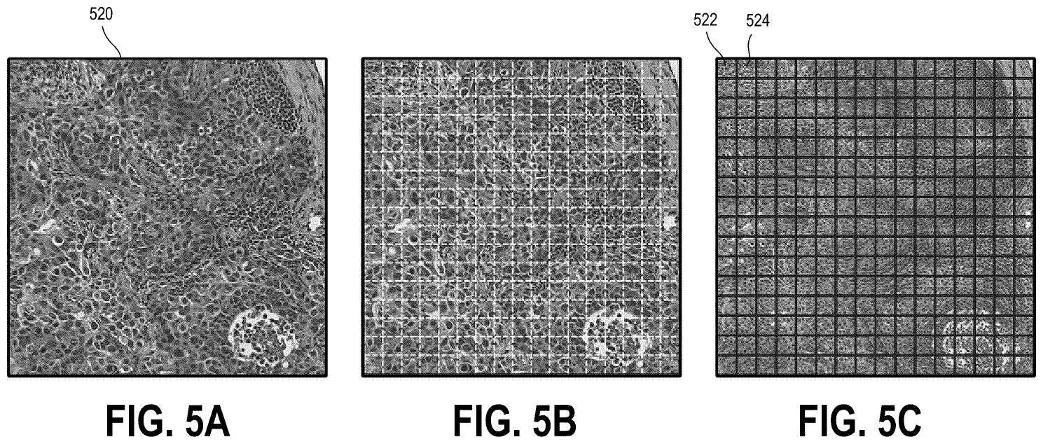

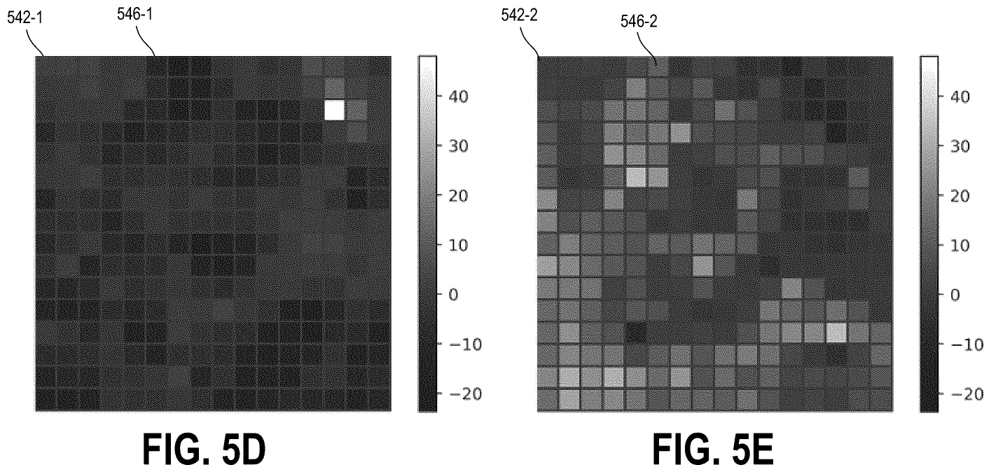

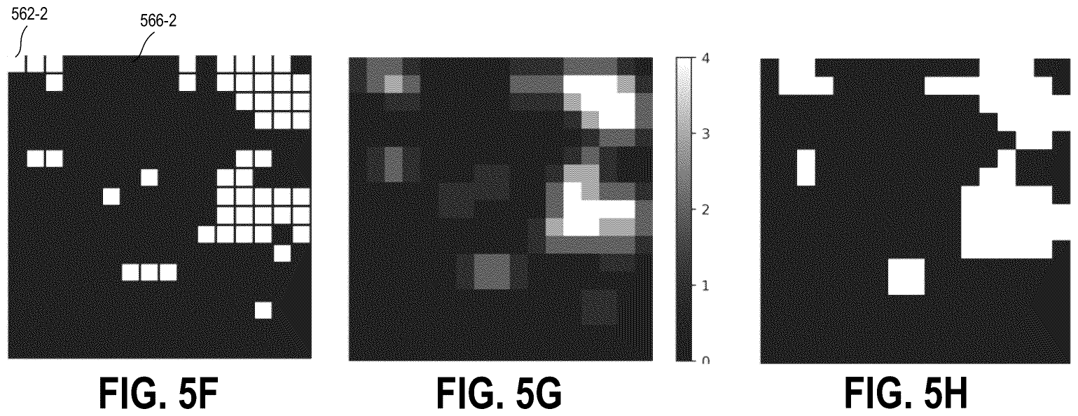

FIGS. 5A, 5B, 5C, 5D, 5E, 5F, 5G, and 5H show an example of identifying at least one TIL in an image of tissue, according to some embodiments of the technology described herein.

FIG. 5A shows an example image 520 of tissue obtained from a subject. In some embodiments, image 520 is obtained using any suitable techniques such as, for example, the techniques described herein including at least with respect to FIGS. 1A and 3A.

FIG. 5B shows broken lines overlayed over the image 520. The broken lines indicate how the image 520 may be cropped to generate sub-images. FIG. 5C shows the resulting sub-images. As shown, the sub-images include sub-image 522 and sub-image 524. In some embodiments, the sub-images are obtained using the techniques described herein, including at least with respect to act 342 of FIG. 3C. For example, the sub-images may be obtained by obtaining crops from the image 520 along the crop lines shown in FIG. 5B. While the example shows that sub-images are obtained for the entire image 520, it should be appreciated that sub-images may be obtained for only a portion of the image 520.

In some embodiments, the sub-images are processed using a neural network model such as any of the neural network models described herein including at least with respect to act 344 of process 340 shown in FIG. 3C and in the section “Neural Networks.”

In some embodiments, the output of the neural network includes, for each sub-image, a sub-image mask with two channels. The first channel is shown in FIG. 5D, and the second channel is shown in FIG. 5E. In the example, the first channel indicates numerical values indicative of the likelihood that the sub-image includes pixels that are part of at least one TIL (“TIL-positive channel”). The second channel indicates numerical values indicative of the likelihood that the sub-image does not include pixels that are part of at least one TIL (“TIL-negative channel”). For example, FIG. 5D shows the first channel of a sub-image mask 542-1 for a first sub-image (e.g., sub-image 522 shown in FIG. 5E). As indicated by the shading, the first channel of the first sub-image mask 542-1 indicates a numerical value indicative of the likelihood that one or more pixels in the first sub-image are part of at least one TIL. FIG. 5E shows the second channel of the first sub-image mask 542-2 for the first sub-image (e.g., sub-image 522 shown in FIG. 5E). As indicated by the shading, the second channel of the first sub-image mask 542-2 indicates a numerical value indicative of the likelihood that one or more pixels of the first sub-image are not part of at least one TIL.

In some embodiments, the numerical values indicated by the channels of the sub-image masks are compared to obtain the mask shown in FIG. 5E. For example, the numerical value indicated by the TIL-positive channel may be compared to the numerical value indicated by the TIL-negative channel. If the numerical value of the TIL-positive channel is greater than that of the TIL-negative channel, then pixels in the sub-image mask may be assigned a first value (e.g., 1) in the mask shown in FIG. 5E. If the numerical value of the TIL-negative channel is greater than that of the TIL-positive channel, then the pixels in sub-image mask may be assigned a second value (e.g., 0) in the mask shown in FIG. 5E. The different values may indicate a lack or presence of at least one TIL in the corresponding sub-image.

For example, the numeric value indicated by the first channel of sub-image mask 542-1 may be compared to the numeric value indicated by the second channel of the sub-image mask 542-2. As shown in FIG. 5F, the resulting value for sub-image mask 562-2 is indicated by a white square, indicating that one or more pixels of the corresponding sub-image (e.g., sub-image 522 shown in FIG. 5C) are part of at least one TIL. The numeric value indicated by the first channel of sub-image mask 546-1 may be compared to the numeric value indicated by the second channel of sub-image mask 546-2. As shown in FIG. 5F, the resulting value for sub-image mask 566-2 is indicated by a black square, indicating that one or more pixels of the corresponding sub-image are not part of at least one TIL.

As shown in FIG. 5G, the mask shown in FIG. 5F may be processed using any suitable image and/or mask processing techniques such as those described herein including at least with respect to act 346 of process 340 shown in FIG. 3C. As shown in the example of FIG. 5G, predictor window summation may be applied to the mask.

In some embodiments, the processed mask (e.g., shown in FIG. 5G) may be binarized to obtain the binarized mask shown in FIG. 5H. For example, values of pixels of the mask shown in FIG. 5G may be compared to a threshold. If a particular value is greater than or equal to a threshold value (e.g., 1, 2, 3, etc.), then the pixel may be assigned a first value (e.g., 1, indicated by white shading). If a particular value is less than the threshold, then the pixel may be assigned a second value (e.g., 0, indicated by black shading).

The mask shown in FIG. 5H may be used to identify portions of the image 520 that include pixels that are part of at least one TIL. For example, the white shaded regions of the mask shown in FIG. 5H may correspond to portions of the image 520 that include pixels that are part of at least one TIL.

### Neural Networks

FIG. 6A illustrates an example architecture of a neural network model 600 which may be used to identify at least one TIL in an image of tissue, according to some embodiments of the technology described herein.

The neural network 600 may receive an input image 602 and process the input image using the layers shown in FIG. 6A to obtain an output indicative of the likelihood that the input image includes at least one TIL. In some embodiments, the neural network includes at least 1 million parameters, at least 5 million parameters, at least 10 million parameters, at least 15 million parameters, at least 20 million parameters, at least 25 million parameters, at least 30 million parameters, at least 35 million parameters, at least 40 million parameters, at least 45 million parameters, at least 50 million parameters, or at least any other suitable number of parameters. In some embodiments, the neural network includes at most 100 million parameters, at most 95 million parameters, at most 90 million parameters, at most 85 million parameters, at most 80 million parameters, at most 75 million parameters, at most 70 million parameters, at most 65 million parameters, at most 60 million parameters, at most 55 million parameters, at most 50 million parameters, or at most any other suitable number of parameters. It should be appreciated that any of the above-listed upper bounds may be coupled with any of the above-listed lower bounds.

As shown in FIG. 6A, the deconvolution portion 604 may be configured to receive an input image 602. The input image 602 may be a sub-image of a whole slide image (WSI) of tissue or an entire WSI image. The input image 602 may be a single channel image or a multi-channel image (e.g., a 3-channel RGB image). When the image 602 is a sub-image of a WSI image, that sub-image may be obtained in any suitable manner. For example, the sub-image may be cropped out of the WSI image.

The input image 602 may have any suitable dimensions, as aspects of the technology described herein are not limited in this respect. For example, input image 602 may have 128×128 pixels per channel, 256×256 pixels per channel, 512×512 pixels per channel, 1024×1024 pixels per channel, 2048×2048 pixels per channel, 4096×4096 pixels per channel, 8192×8192 pixels per channel, or any other suitable number of pixels per channel.

In some embodiments, prior to being provided as input to the deconvolution portion 604, the input image 602 is processed according to any suitable processing technique such as, for example, the image processing techniques described herein including at least with respect to FIG. 3A.

In some embodiments, the image 602 is provided as input to the deconvolution portion 604. The deconvolution portion 604 includes a 2D convolutional layer 604-1. As an example, the 2D convolutional layer may have 3 input channels and 3 output channels, a kernel size of 1, a stride of 1, and a padding of 0. In some embodiments, the deconvolution portion 604 is configured to convert the image 602 to an edge map using holistically-nested edge detection (HED). HED is described by Xie, S. and Zhuowen, T. (“Holistically-nested edge detection.” Proceedings of the IEEE international conference on computer vision. 2015), which is incorporated by reference herein in its entirety.

In some embodiments, the output of the deconvolution portion 604 is coupled to neural network portion 650. For example, the output of the deconvolution portion 604 may be coupled to the neural network portion 650 in a feedforward manner. For example, the output of the first two channels of the deconvolution portion 604 may be forwarded to the input of neural network portion 650. In some embodiments, the neural network portion 650 may be implemented using architecture of a convolutional neural network (CNN) such as, for example, EfficientNet, EfficientNetV2, ResNet, VGGNet, DenseNet, or any other suitable type of CNN, as aspects of the technology described herein are not limited in this respect. For example, the neural network portion 650 may be implemented using the EfficientNetV2-M architecture. The EfficientNet architecture is described by Tan, M., and Le, Q. (“Efficientnet: Rethinking model scaling for convolutional neural networks.” International conference on machine learning. PMLR, 2019), which is incorporated by reference herein in its entirety. The EfficientNetV2 architecture is described by Tan, M. and Lee, Q. (“EfficientNetV2: Smaller Models and Faster Training.” International conference on machine learning. PMLR, 2021), which is incorporated by reference herein in its entirety. The ResNet architecture is described by He, K., et al. (“Deep residual learning for image recognition.” Proceedings of the IEEE conference on computer vision and pattern recognition. 2016), which is incorporated by reference herein in its entirety. The VGGNet architecture is described by Simonyan, K., and Zisserman, A. (“Very deep convolutional networks for large-scale image recognition.” arXiv preprint arXiv:1409.1556 (2014)), which is incorporated by reference herein in its entirety. The DenseNet architecture is described by Huang, Gao, et al. (“Densely connected convolutional networks. arXiv. org.” (2016)), which is incorporated by reference herein its entirety.

In some embodiments, neural network portion 650 includes: an adapter portion 606, convolution and batch normalization layer 608, FusedBMConv layers 610-614, inverted residual layers 616, 616, 620, and classification portion 622.

In some embodiments, the output of the deconvolution portion 604 is coupled to the adapter portion 606. For example, the output of the deconvolution portion 604 may be coupled to the adapter portion 606 in a feedforward manner. The adapter portion 606 includes: a 2D convolutional layer 606-1, batch normalization layer 606-2, and activation layer 606-3. The activation layer 606-3 may use any suitable activation function (e.g., sigmoid linear unit (SiLU), rectified linear unit (ReLU), leaky ReLU, hyperbolic, softmax, etc.).

In some embodiments, the output of the adapter portion 606 is coupled to the convolution and batch normalization layers 608. For example, the output of the adapter portion 606 may be coupled to the convolution and batch normalization layer 608 in a feedforward manner.

In some embodiments, the output of the convolution and batch normalization layers 608 is coupled to the Fused-MBConv layer 610. Fused-MBConv layers are described by Gupta, S. and Tan, M. (“Creating accelerator-optimized neural networks with AutoML.” Google AI Blog 2.1 (2019)), which is incorporated by reference herein in its entirety. In some embodiments, the Fused-MBConv layers 610, 612, 614 are connected using residual connections.

In some embodiments, the output of the fused-MBConv layer 614 is coupled to the inverted residual layer 616. Inverted residual layers are described by Sandler, M., et al. (“Mobilenetv2: Inverted residuals and linear bottlenecks.” CVPR, 2018) and Tan, M. and Le, Q. (“Efficientnet: Rethinking model scaling for convolutional neural networks.” ICML, 2019a), each of which is incorporated by reference herein in its entirety. In some embodiments, the inverted residual layers are connected using residual connections.

In some embodiments, the output of the inverted residual layer 620 is coupled to the classification portion 622. The classification portion 622 includes: a convolutional layer 622-1, batch normalization layer 622-2, activation layer 622-3, global pooling layer 622-4, and fully connected layer 622-5. The activation layer 622-3 may use any suitable activation function (e.g., sigmoid linear unit (SiLU), rectified linear unit (ReLU), leaky ReLU, hyperbolic, softmax, etc.).

In some embodiments, the classification portion 622 is configured to provide output 624. In some embodiments, the output is indicative of a likelihood that the input image 602 includes pixels that are part of at least one TIL. The output may include any suitable number of channels, as aspects of the technology described herein are not limited in this respect. For example, the output may include 1 channel, at least 2 channels, at least 3 channels, or at least any other suitable number of channels. For example, the output may include 2 channels; the first channel may include a numeric value indicative of a likelihood that the input image 602 includes pixels that are part of at least one TIL, and the second channel may include a numeric value indicative of a likelihood that the input image does not include pixels that are part of at least one TIL.

### Training

FIG. 6B is a flowchart of an illustrative process 660 for training a neural network model to predict, for one or more pixels in an image, respective numeric values indicative of the likelihood that the particular pixel(s) are part of the TIL. One or more acts (e.g., all acts) of process 300 may be performed automatically by any suitable computing device(s). For example, the act(s) may be performed by a laptop computer, a desktop computer, one or more servers, in a cloud computing environment, computing device 900 as described herein including at least with respect to FIG. 9, and/or in any other suitable way.

At act 662, a set of images is obtained. In some embodiments, each of at least some (e.g., all) of the images in the set of images includes an image of tissue from subjects having cancer. For example, the set of images may include images of tissue from subjects having cancers where TLS and/or TIL is of interest. In some embodiments, all of the images in the set of images are images of tissue from subjects having the same type of cancer. In some embodiments, different subsets of images in the set of images are images of tissue from subjects having different types of cancer.

In some embodiments, some or all of the images in the set of images may be obtained or may have been previously-obtained using an imaging platform such as, for example, imaging platform 104 described with reference to FIG. 1A. Each of the images may be a single-channel image or multi-channel image (e.g., a 3-channel RGB image). For example, an image may be a whole slide image (WSI). The image may have any suitable dimensions, as aspects of the technology are not limited in this respect. For example, an image may have at least 200×200 pixels values per channel, 250×250 pixel values per channel, 300×300 pixel values per channel, 350×350 pixel values per channel, 400×400 pixel values per channel, 450×450 pixel values per channel, 500×500 pixel values per channel, 550×550 pixel values per channel, 600×600 pixel values per channel, 650×650 pixel values per channel, 700×700 pixel values per channel, 750×750 pixel values per channel, 800×800 pixel values per channel, 850×850 pixel values per channel, 900×900 pixel values per channel, 950×950 pixel values per channel, 1,000×1,000 pixel values per channel, or any other suitable number of pixel values per channel. The dimensions of the image may be within any suitable range such as, for example, 128-3,000×128-3,000 pixel values per channel, 200-2,000×200-2,000 pixel values per channel, 300-1,000×300-1,000 pixel values per channel, 400-800×400-800 pixel values per channel, or any other suitable range within these ranges.

In some embodiments, the set of images may include any suitable number of images, as aspects of the technology described herein are not limited in this respect. For example, the set of images may include at least 1,000 images, at least 10,000 images, at least 20,000 images, at least 30,000 images, at least 40,000 images, at least 50,000 images, at least 60,000 images, at least 70,000 images, at least 80,000 images, at least 90,000 images, at least 100,000 images, at least 110,000 images, at least 120,000 images, at least 130,000 images, at least 140,000 images, at least 150,000 images, at least 175,000 images, at least 200,000 images, at least 250,000 images, or at least any other suitable number of images. In some embodiments, the set of images may include at most 400,000 images, at most 350,000 images, at most 300,000 images, at most 250,000 images, at most 200,000 images, at most 175,000 images, at most 150,000 images, at most 125,000 images, or at most any other suitable number of images. It should be appreciated that any of the above-listed upper bounds may be coupled with any of the above-listed lower bounds.

In some embodiments, the obtained images may be augmented using any suitable augmentation techniques. As a nonlimiting example, in some embodiments, one or more of the obtained images may be augmented using random rotations (e.g., by a random number of degrees including 90 degrees and multiples thereof). In some embodiments, one or more of the images may be flipped horizontally or vertically. In some embodiments, a brightness of one or more of the images may be adjusted (e.g., randomly). In some embodiments, a contrast of one or more of the images may be adjusted (e.g., randomly). In some embodiments, channel shuffling may be applied to one or more of the images.

At act 664, sub-images are generated from the obtained images. The sub-images may be used for training and/or testing, as described herein. In some embodiments, a sub-image may be obtained from an image in the set of images obtained at act 662. For example, the sub-image may be cropped out of the image. The dimensions of the sub-image may be smaller than the corresponding dimensions of the image from which the sub-image is obtained. For example, the sub-image may have at least 128×128 pixels per channel, at least 256×256 pixels per channel, at least 512×512 pixels per channel, or any other suitable number of pixels per channel. The sub-image may have at most 2048×2048 pixels per channel, at most 1024×1024 pixels per channel, at most 512×512 pixels per channel, or any other suitable number of pixels per channel. It should be appreciated that any of the above-listed upper bounds may be coupled with any of the above-listed lower bounds.

At act 666, the sub-images are labeled. In some embodiments, the label is a binary label. For example, the label may indicate whether or not the sub-image includes at least one TIL. The sub-images may be labeled using any suitable technique, as aspects of the technology described herein are not limited in this respect. For example, in some embodiments, one or more of the sub-images may be labeled manually (e.g., by a user). Additionally, or alternatively, in some embodiments, the images obtained at act 662 may have been previously labeled.

In some embodiments, to prevent bias due to imbalanced data, random resampling is applied to the labeled sub-images. The random resampling may be performed using any suitable techniques, as aspects of the technology described herein are not limited in this respect. For example, the random resampling may be performed using the Imbalanced Learn RandomOverSampler. After resampling, the sub-images may be split into train and test sets for training and testing the neural network model, as described herein.

At act 668, a neural network is trained using the generated sub-images to obtain a trained neural network model. In some embodiments, for each epoch, any suitable number of sub-images may be used to train the neural network model and any suitable number of sub-images may be used to validate the neural network model. In some embodiments, the neural network model may be trained and validated with any suitable number of epochs, as aspects of the technology described herein are not limited in this respect. For example, the neural network may be trained with at least 30 epochs, at least 35 epochs, at least 40 epochs, at least 45 epochs, at least 50 epochs, at least 55 epochs, at least 60 epochs, at least 65 epochs, at least 70 epochs, at least 75 epochs, at least 80 epochs, at least 85 epochs, at least 90 epochs, or any other suitable number of epochs. In some embodiments, the neural network model may be trained with between 20 and 100 epochs, between 30 and 90 epochs, between 40 and 80 epochs, between 45 and 75 epochs, between 50 and 65 epochs, or any suitable number of epochs within any of the above-listed ranges.

In some embodiments, training the neural network model at act 668 includes training the neural network model using any suitable optimizer. For example, in some embodiments, training the neural network may include using the Optuna optimizer described by Akiba, T., et al. (“Optuna: A next-generation hyperparameter optimization framework.” Proceedings of the 25th ACM SIGKDD international conference on knowledge discovery & data mining. 2019), which is incorporated by reference herein in its entirety. In some embodiments, training the neural network model may include using the Adam optimizer described by Kingma, D. and Jimmy, B. (“Adam: A method for stochastic optimization.” arXiv preprint arXiv:1412.6980 (2014)), which is incorporated by reference herein in its entirety. In some embodiments, training the neural network model may include using the SGD Momentum optimizer described by Duda, J. (“SGD momentum optimizer with step estimation by online parabola model.” arXiv preprint arXiv:1907.07063 (2019)), which is incorporated by reference herein in its entirety. In some embodiments, training the neural network model may include using the AdaBound optimizer described by Luo, L., et al. (“Adaptive gradient methods with dynamic bound of learning rate.” arXiv preprint arXiv:1902.09843 (2019)), which is incorporated by reference herein in its entirety. In some embodiments, training the neural network model may include using the AdamW optimizer described by Loshchilov, I., and Hutter, F. (“Decoupled weight decay regularization.” arXiv preprint arXiv:1711.05101 (2017)), which is incorporated by reference herein in its entirety.

In some embodiments, one or more initial parameters are selected for training the neural network model. For example, an initial learning rate may be selected for the neural network model. It should be appreciated that the initial learning rate may be selected to be any suitable initial learning rates, as aspects of the technology described herein are not limited in this respect. As a nonlimiting example, the initial learning rate may be set to 5.69×10⁻⁵. In some embodiments, during training, the learning rate is adjusted based on a learning rate scheduler. The learning rate scheduler may include any suitable learning rate scheduler, as aspects of the technology described herein are not limited in this respect. For example, cosine annealing may be used. Cosine annealing is described by L., Ilya, and Hutter, F. (“Sgdr: Stochastic gradient descent with warm restarts.” arXiv preprint arXiv:1608.03983 (2016). For example, the cosine annealing warm restarts learning rate scheduler may be used with the parameters of T=0, T_mult=1, eta_max=1×10⁻⁹, T_up=10, and gamma=0.5.

In some embodiments, one or more initial weights are selected for the deconvolution portion (e.g., deconvolution portion 604) of the neural network model. The initial weights may be precomputed. For example, the initial weights may be: [[0.65, 0.7, 0.29], [0.07, 0.99, 0.11], [0.0, 0.0, 0.0]]. In some embodiments, the initial weights of the deconvolution portion of the neural network model may be optimized during training at act 668.

In some embodiments, gradient scaling is performed during the training of the neural network at act 668. Gradient scaling may be performed using the PyTorch GradScaler, for example.

In some embodiments, training the neural network at act 668 includes minimizing a loss function. The loss function may include any suitable loss function, as aspects of the technology described herein are not limited in this respect. In some embodiments, the loss function may account for cross-entropy loss.

### EXAMPLES

This example shows that identifying characteristics of TLSs according to embodiments of the technology described herein can be used for clinical decision making and to predict therapeutic response. This example includes the following sections: “Materials and Methods” and “Results.” In this example, a portion of an image that include TILs may be referred to as “lymphocyte immune infiltrated area (LIIA).”

### Materials and Methods

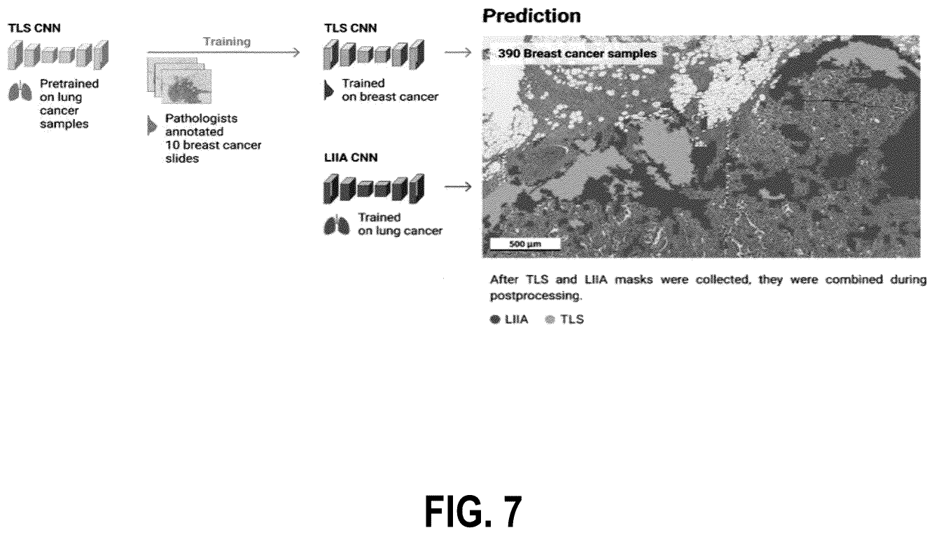

A cohort of 445 The Cancer Genome Atlas (TCGA) breast cancer cases was divided into Luminal (n=192) with histological subtype Invasive Lobular Carcinoma (ILC), HER2-enriched (n=110) and basal-like (n=143) molecular subtypes. The hematoxylin and eosin (H&E) stain whole slide images (WSIs) were retrieved with clinical annotations and outcomes. A combination of convolutional neural network (CNN)-based deep learning models was used to detect and classify intratumoral and borderline TLS, lymphocyte immune infiltrated area (LIIA) and tumor zone segmentation. As shown in FIG. 7, a TLS CNN model was used to generate a TLS mask and a LIIA CNN was used to generate a TIL mask. Models were validated on the holdout dataset and later visually assessed on separately chosen whole slides by two experienced pathologists. TLS and tumor zone models' predictions were combined to specify the location of TLS. The CNNs were trained on the TCGA dataset and verified it by the Kaplan-Meier estimate and Cox regression analyses. Correlations were calculated with the Spearman test.

### Results

390 samples (87%) were eligible for CNN analysis. 55 samples (13%) were excluded from analysis due to artifacts or incomplete clinical annotations. TLS (intratumoral and borderline) were detected in 53% (n=207) of the samples, with the highest score of 28 TLS per mm² (Q3=8.12 TLS/mm²). In subgroup analysis, TLS were detected in 51% of the Her2-enriched subtype samples, 37% of the Luminal subtype samples, and 74% of the Basal-like subtype samples. TLS density (per mm²) was significantly higher (p-value<0.0001, Mann-Whitney test) in the Basal-like subtype samples (Q3=14.2, Q2=2.2 TLS/mm2) compared to the Luminal subtype (Q3=3, Q2=0, TLS/mm2) and HER2 enriched subtype (Q3=5.6, Q2=0.2, TLS/mm2) samples.

There is an association between LIIA and tumor mutational burden (TMB)-high samples (TMB>10, p-value<0.0001), but not between TLS and TMB. There was no association observed between microsatellite instability (MSI)-high status and calculated metrics.

There was a significant correlation of PD-L1 gene expression and predicted LIIA in Basal-like (r=0.31) and HER2 enriched subtypes (r=0.46), p-value<0.01. No correlation was found in Luminal subtype. PD-L1 expression had positive correlation with TLS density in the Basal-like subtype (r=0.25, p-value <0.01) and the Luminal subtype (r=0.18, p-value <0.05). No correlation was observed for the HER2-enriched subtype.

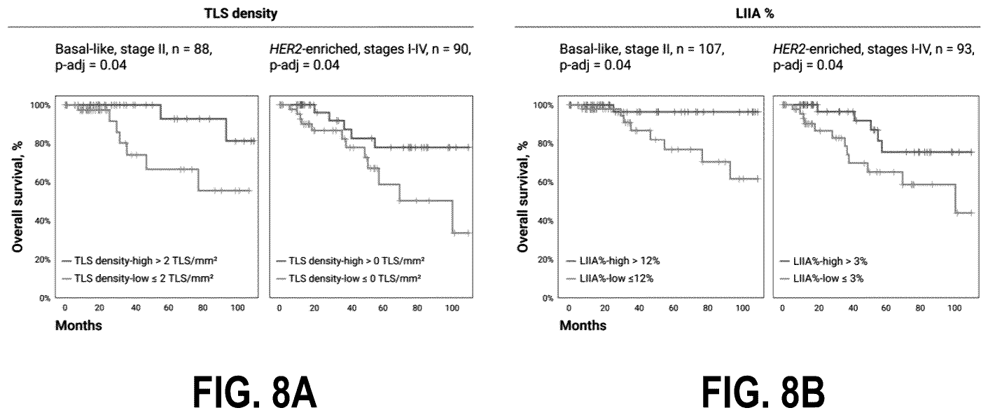

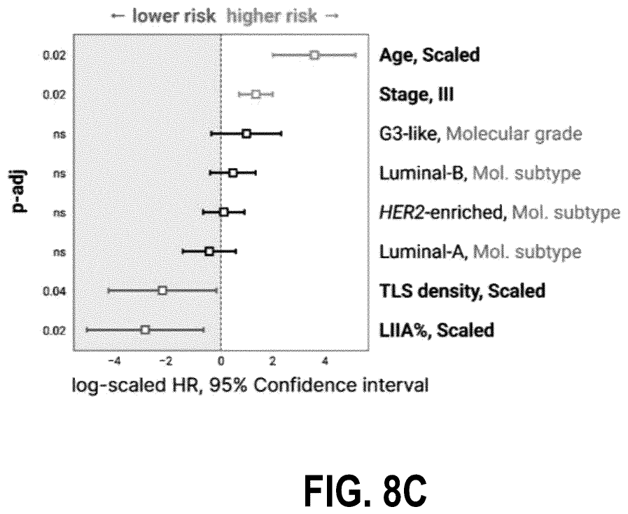

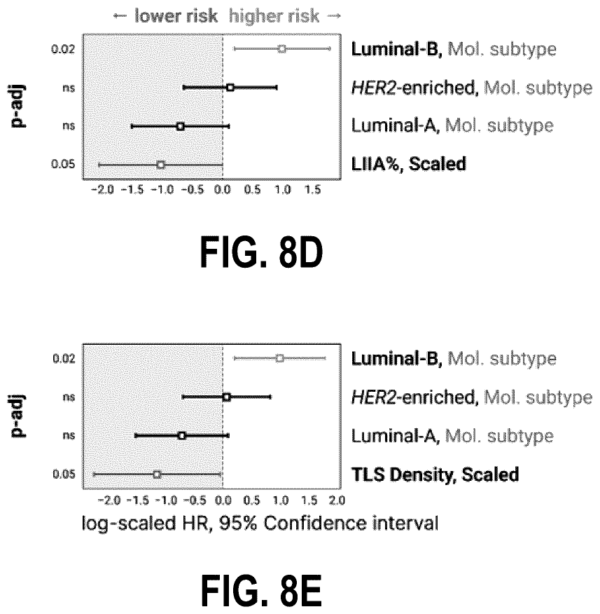

FIG. 8A is an example showing that overall survival improves when the density of TLSs in the tissue is greater than a threshold, according to some embodiments of the technology described herein. In the Basal-like subtype, Stage II patients with ≥2.2 TLS/mm² (Q2), had a significantly better overall survival (OS) (p-value <0.001) for 10 years; 5-year survival was 93±7% for the TLS-high group and 48±16% for the TLS-low group. In the HER2-enriched subtype, patients with ≥0.2 TLS/mm² had significantly higher OS (p-value <0.05) for 10 years; 5-year survival was 75±8% for TLS-high group and 58±12% for the TLS-low group.

FIG. 8B is an example showing that overall survival improves when the percentage of TILs in the tissue is greater than a threshold, according to some embodiments of the technology described herein. A higher percentage of LIIA was observed to be positively associated with OS in the Basal-like subtype (p-value=0.05, Q3=20%) and HER2-enriched subtype (p-value<0.05, Q2=3%).

Multivariable analysis was performed. FIGS. 8C, 8D, and 8E show the result of the analysis. After adjusting age, stage, and grade, LIIA and TLS density were found to be significant independent positive prognostic factors for overall survival in the Basal-like subtype (LIIA—hazard ratio (HR): 0.02, standard error (SE): 1.34, p-value<0.005; for high TLS group HR: 0.09, SE: 0.76, p-value<0.005). For the HER2 enriched subtype, TLS density was also a significant predictor (HR: 0.05, SE: 1.42, p-value<0.05), and LIIA was positively correlated (HR: 0.0002, SE: 4.82). In the Luminal subtype (ILC), both characteristics were not significant and didn't impact the survival outcome. All models had global p-value <0.0005 and concordance index >0.86.

### Computer Implementation

An illustrative implementation of a computer system 900 that may be used in connection with any of the embodiments of the technology described herein (e.g., such as the process 300 of FIG. 3A, process 330 of FIG. 3B, and/or process 340 of FIG. 3C) is shown in FIG. 9. The computer system 900 includes one or more processors 910 and one or more articles of manufacture that comprise non-transitory computer-readable storage media (e.g., memory 920 and one or more non-volatile storage media 930). The processor 910 may control writing data to and reading data from the memory 920 and the non-volatile storage device 930 in any suitable manner, as the aspects of the technology described herein are not limited to any particular techniques for writing or reading data. To perform any of the functionality described herein, the processor 910 may execute one or more processor-executable instructions stored in one or more non-transitory computer-readable storage media (e.g., the memory 920), which may serve as non-transitory computer-readable storage media storing processor-executable instructions for execution by the processor 910.

Computing device 900 may include a network input/output (I/O) interface 940 via which the computing device may communicate with other computing devices. Such computing devices may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Computing device 900 may also include one or more user I/O interfaces 950, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone, a tablet, or any other suitable portable or fixed electronic device.

The above-described embodiments can be implemented in any of numerous ways. For example, the embodiments may be implemented using hardware, software, or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-described functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-described functions of one or more embodiments. The computer-readable medium may be transportable such that the program stored thereon can be loaded onto any computing device to implement aspects of the techniques described herein. In addition, it should be appreciated that the reference to a computer program which, when executed, performs any of the above-described functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques described herein.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code can be executed on any suitable processor or collection of processors, whether provided in a single computer or distributed among multiple computers.

The foregoing description of implementations provides illustration and description but is not intended to be exhaustive or to limit the implementations to the precise form disclosed. Modifications and variations are possible in light of the above teachings or may be acquired from practice of the implementations. In other implementations the methods depicted in these figures may include fewer operations, different operations, differently ordered operations, and/or additional operations. Further, non-dependent blocks may be performed in parallel.

It will be apparent that example aspects, as described above, may be implemented in many different forms of software, firmware, and hardware in the implementations illustrated in the figures.

### Biological Samples

Any of the methods, systems, or other claimed elements may use or be used to analyze a biological sample from a subject. In some embodiments, a biological sample is obtained from a subject having, suspected of having cancer, or at risk of having cancer. In some embodiments, the biological sample is a sample of a tumor from a subject. In some embodiments, the biological sample is a sample of tissue from a subject.

A sample of a tumor, in some embodiments, refers to a sample comprising cells from a tumor. In some embodiments, the sample of the tumor comprises cells from a benign tumor, e.g., non-cancerous cells. In some embodiments, the sample of the tumor comprises cells from a premalignant tumor, e.g., precancerous cells. In some embodiments, the sample of the tumor comprises cells from a malignant tumor, e.g., cancerous cells. Examples of tumors include, but are not limited to, adenomas, fibromas, hemangiomas, lipomas, cervical dysplasia, metaplasia of the lung, leukoplakia, carcinoma, sarcoma, germ cell tumors, and blastoma.

A sample of a tissue, in some embodiments, refers to a sample comprising cells from a tissue. In some embodiments, the sample of the tumor comprises non-cancerous cells from a tissue. In some embodiments, the sample of the tumor comprises precancerous cells from a tissue.

Methods of the present disclosure encompass a variety of tissue including organ tissue or non-organ tissue, including but not limited to, muscle tissue, brain tissue, lung tissue, liver tissue, epithelial tissue, connective tissue, and nervous tissue. In some embodiments, the tissue may be normal tissue, or it may be diseased tissue, or it may be tissue suspected of being diseased. In some embodiments, the tissue may be sectioned tissue or whole intact tissue. In some embodiments, the tissue may be animal tissue or human tissue. Animal tissue includes, but is not limited to, tissues obtained from rodents (e.g., rats or mice), primates (e.g., monkeys), dogs, cats, and farm animals.

The biological sample may be from any source in the subject's body including, but not limited to, skin (including portions of the epidermis, dermis, and/or hypodermis), oropharynx, laryngopharynx, esophagus, stomach, bronchus, salivary gland, tongue, oral cavity, nasal cavity, vaginal cavity, anal cavity, bone, bone marrow, brain, thymus, spleen, small intestine, appendix, colon, rectum, anus, liver, lung, lung, biliary tract, pancreas, kidney, ureter, bladder, urethra, uterus, vagina, vulva, ovary, cervix, scrotum, penis, prostate, testicle, seminal vesicles, and/or any type of tissue (e.g., muscle tissue, adipose tissue, epithelial tissue, connective tissue, or nervous tissue).

Any of the biological samples described herein may be obtained from the subject using any known technique. See, for example, the following publications on collecting, processing, and storing biological samples, each of which are incorporated by reference herein in its entirety: Biospecimens and biorepositories: from afterthought to science by Vaught et al. (Cancer Epidemiol Biomarkers Prev. 2012 February; 21(2):253-5), and Biological sample collection, processing, storage and information management by Vaught and Henderson (IARC Sci Publ. 2011; (163):23-42).

In some embodiments, the biological sample may be obtained from a surgical procedure (e.g., laparoscopic surgery, microscopically controlled surgery, or endoscopy), bone marrow biopsy, punch biopsy, endoscopic biopsy, or needle biopsy (e.g., a fine-needle aspiration, core needle biopsy, vacuum-assisted biopsy, or image-guided biopsy).

In some embodiments, one or more than one cell (i.e., a cell biological sample) may be obtained from a subject using a scrape or brush method. The cell biological sample may be obtained from any area in or from the body of a subject including, for example, from one or more of the following areas: the cervix, esophagus, stomach, bronchus, or oral cavity. In some embodiments, one or more than one piece of tissue (e.g., a tissue biopsy) from a subject may be used. In certain embodiments, the tissue biopsy may comprise one or more than one (e.g., 2, 3, 4, 5, 6, 7, 8, 9, 10, or more than 10) biological samples from one or more tumors or tissues known or suspected of having cancerous cells.

Any of the biological samples from a subject described herein may be stored using any method that preserves stability of the biological sample. In some embodiments, preserving the stability of the biological sample means inhibiting components (e.g., DNA, RNA, protein, or tissue structure or morphology) of the biological sample from degrading until they are measured so that when measured, the measurements represent the state of the sample at the time of obtaining it from the subject. In some embodiments, a biological sample is stored in a composition that is able to penetrate the same and protect components (e.g., DNA, RNA, protein, or tissue structure or morphology) of the biological sample from degrading. As used herein, degradation is the transformation of a component from one from to another such that the first form is no longer detected at the same level as before degradation.

In some embodiments, a biological sample (e.g., tissue sample) is fixed. As used herein, a “fixed” sample relates to a sample that has been treated with one or more agents or processes in order to prevent or reduce decay or degradation, such as autolysis or putrefaction, of the sample. Examples of fixative processes include but are not limited to heat fixation, immersion fixation, and perfusion. In some embodiments a fixed sample is treated with one or more fixative agents. Examples of fixative agents include but are not limited to cross-linking agents (e.g., aldehydes, such as formaldehyde, formalin, glutaraldehyde, etc.), precipitating agents (e.g., alcohols, such as ethanol, methanol, acetone, xylene, etc.), mercurials (e.g., B-5, Zenker's fixative, etc.), picrates, and Hepes-glutamic acid buffer-mediated organic solvent protection effect (HOPE) fixatuve. In some embodiments, a biological sample (e.g., tissue sample) is treated with a cross-linking agent. In some embodiments, the cross-linking agent comprises formalin. In some embodiments, a formalin-fixed biological sample is embedded in a solid substrate, for example paraffin wax. In some embodiments, the biological sample is a formalin-fixed paraffin-embedded (FFPE) sample. Methods of preparing FFPE samples are known, for example as described by Li et al. JCO Precis Oncol. 2018; 2: PO.17.00091.

Non-limiting examples of preservants include formalin solutions, formaldehyde solutions, RNALater or other equivalent solutions, TriZol or other equivalent solutions, DNA/RNA Shield or equivalent solutions, EDTA (e.g., Buffer AE (10 mM Tris-Cl; 0.5 mM EDTA, pH 9.0)) and other coagulants, and Acids Citrate Dextronse (e.g., for blood specimens). In some embodiments, special containers may be used for collecting and/or storing a biological sample. For example, a vacutainer may be used to store blood. In some embodiments, a vacutainer may comprise a preservant (e.g., a coagulant, or an anticoagulant). In some embodiments, a container in which a biological sample is preserved may be contained in a secondary container, for the purpose of better preservation, or for the purpose of avoid contamination.

Any of the biological samples from a subject described herein may be stored under any condition that preserves stability of the biological sample. In some embodiments, the biological sample is stored at a temperature that preserves stability of the biological sample. In some embodiments, the sample is stored at room temperature (e.g., 25° C.). In some embodiments, the sample is stored under refrigeration (e.g., 4° C.). In some embodiments, the sample is stored under freezing conditions (e.g., −20° C.). In some embodiments, the sample is stored under ultralow temperature conditions (e.g., −50° C. to −800° C.). In some embodiments, the sample is stored under liquid nitrogen (e.g., −1700° C.). In some embodiments, a biological sample is stored at −60° C. to −80° C. (e.g., −70° C.) for up to 5 years (e.g., up to 1 month, up to 2 months, up to 3 months, up to 4 months, up to 5 months, up to 6 months, up to 7 months, up to 8 months, up to 9 months, up to 10 months, up to 11 months, up to 1 year, up to 2 years, up to 3 years, up to 4 years, or up to 5 years). In some embodiments, a biological sample is stored as described by any of the methods described herein for up to 20 years (e.g., up to 5 years, up to 10 years, up to 15 years, or up to 20 years).

In some embodiments, obtaining the biological sample may include: (1) collecting tissue from a subject; (2) fixing the tissue (e.g., using FFPE preparation); (3) placing portions of the fixed tissue on one or more slides and staining the tissue; and (4) imaging the slides to produce one or more images of the tissue (e.g., whole slide images).

In some embodiments, the tissue may be collected using any suitable method such as a biopsy (e.g., excisional biopsy) or surgical resection. For example, the tissue may be obtained by skin excision with melanoma, breast tissue excision with carcinoma, liver tissue excision with carcinoma, muscles tissue excision with leiomyosarcoma. The type of biopsy and the location of the tissue being biopsied or resected may be determined by the type of tumor, tumor localization and tumor stage.

In some embodiments, after the tissue sample has been collected, the tissue sample may be fixed in formalin to preserve its structure, for example, using formalin-fixed paraffin-embedded (FFPE) tissue preparation techniques described herein. In some embodiments, the tissue may be dehydrated and then embedded in paraffin wax and cut into thin sections (e.g., 4-5 micrometers) using a microtome.

In some embodiments, the thin tissue sections may be placed on slides and stained with hematoxylin and eosin (H&E) stain. The H&E stain allows for the visualization of different tissue components such as nuclei, cytoplasm, and extracellular matrix. The H&E stain may be applied in stages. The tissue sections may be stained with hematoxylin, which binds to the basic structures in the tissue, such as the nuclei, and turns them blue. The tissue sections may then be washed and stained with eosin, which binds to acidic structures, such as the cytoplasm, and turns them pink.

In some embodiments, the H&E stained tissue sections on the slides may be imaged using a slide scanner. A slide scanner may be any suitable high-resolution digital microscope that captures high-quality images of the tissue sections. The images so obtained may then be analyzed including by using the neural network techniques described herein to identify tertiary lymphoid structures and obtain information about any such identified structures.

### Subjects

Aspects of this disclosure relate to a biological sample that has been obtained from a subject. In some embodiments, a subject is a mammal (e.g., a human, a mouse, a cat, a dog, a horse, a hamster, a cow, a pig, or other domesticated animal). In some embodiments, a subject is a human. In some embodiments, a subject is an adult human (e.g., of 18 years of age or older). In some embodiments, a subject is a child (e.g., less than 18 years of age). In some embodiments, a human subject is one who has or has been diagnosed with at least one form of cancer.

In some embodiments, a cancer from which a subject suffers is a carcinoma (e.g., squamous cell carcinoma, basal cell carcinoma, adenocarcinoma, transitional cell carcinoma, etc., of different localizations such as cervix, lung, head & neck, skin, stomach, intestine, colon, rectum, liver, pancreas), a sarcoma, or a myeloma. Carcinoma refers to a malignant neoplasm of epithelial origin or cancer of the internal or external lining of the body. Sarcoma refers to cancer that originates in supportive and connective tissues such as bones, tendons, cartilage, muscle, and fat. Myeloma is cancer that originates in the plasma cells of bone marrow.

In some embodiments, a subject is at risk for developing cancer, e.g., because the subject has one or more genetic risk factors, or has been exposed to or is being exposed to one or more carcinogens (e.g., cigarette smoke, or chewing tobacco).

### Methods of Treatment

In certain methods described herein, an effective amount of anti-cancer therapy described herein may be administered or recommended for administration to a subject (e.g., a human) in need of the treatment via a suitable route (e.g., intravenous administration).

The subject to be treated by the methods described herein may be a human patient having, suspected of having, or at risk for a cancer. Examples of a cancer are provided herein. At the time of diagnosis, the cancer may be cancer of unknown primary. The subject to be treated by the methods described herein may be a mammal (e.g., may be a human).

A subject having a cancer may be identified by routine medical examination, e.g., laboratory tests, biopsy, PET scans, CT scans, or ultrasounds. A subject suspected of having a cancer might show one or more symptoms of the disorder, e.g., unexplained weight loss, fever, fatigue, cough, pain, skin changes, unusual bleeding or discharge, and/or thickening or lumps in parts of the body. A subject at risk for a cancer may be a subject having one or more of the risk factors for that disorder. For example, risk factors associated with cancer include, but are not limited to, (a) viral infection (e.g., herpes virus infection), (b) age, (c) family history, (d) heavy alcohol consumption, (e) obesity, and (f) tobacco use.

The dosage of anti-cancer therapy administered to a subject may vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner.

Empirical considerations, such as the half-life of a therapeutic compound, generally contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy and is generally (but not necessarily) based on treatment, and/or suppression, and/or amelioration, and/or delay of a cancer. Alternatively, sustained continuous release formulations of an anti-cancer therapeutic agent may be appropriate. Various formulations and devices for achieving sustained release are known in the art.

In some embodiments, dosages for an anti-cancer therapeutic agent as described herein may be determined empirically in individuals who have been administered one or more doses of the anti-cancer therapeutic agent. Individuals may be administered incremental dosages of the anti-cancer therapeutic agent. To assess efficacy of an administered anti-cancer therapeutic agent, one or more aspects of a cancer (e.g., tumor formation, tumor growth, molecular category identified for the cancer using the techniques described herein) may be analyzed.

For the purpose of the present disclosure, the appropriate dosage of an anti-cancer therapeutic agent will depend on the specific anti-cancer therapeutic agent(s) (or compositions thereof) employed, the type and severity of cancer, whether the anti-cancer therapeutic agent is administered for preventive or therapeutic purposes, previous therapy, the patient's clinical history and response to the anti-cancer therapeutic agent, and the discretion of the attending physician. Typically, the clinician will administer an anti-cancer therapeutic agent, such as an antibody, until a dosage is reached that achieves the desired result.

Administration of an anti-cancer therapeutic agent can be continuous or intermittent, depending, for example, upon the recipient's physiological condition, whether the purpose of the administration is therapeutic or prophylactic, and other factors known to skilled practitioners. The administration of an anti-cancer therapeutic agent (e.g., an anti-cancer antibody) may be essentially continuous over a preselected period of time or may be in a series of spaced dose, e.g., either before, during, or after developing cancer.

As used herein, the term “treating” refers to the application or administration of a composition including one or more active agents to a subject, who has a cancer, a symptom of a cancer, or a predisposition toward a cancer, with the purpose to cure, heal, alleviate, relieve, alter, remedy, ameliorate, improve, or affect the cancer or one or more symptoms of the cancer, or the predisposition toward a cancer.

Alleviating a cancer includes delaying the development or progression of the disease or reducing disease severity. Alleviating the disease does not necessarily require curative results. As used therein, “delaying” the development of a disease (e.g., a cancer) means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given period and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detected and assessed using clinical techniques known in the art. However, development also refers to progression that may be undetectable. For purpose of this disclosure, development or progression refers to the biological course of the symptoms. “Development” includes occurrence, recurrence, and onset. As used herein “onset” or “occurrence” of a cancer includes initial onset and/or recurrence.

Conventional methods, known to those of ordinary skill in the art of medicine, may be used to administer the anti-cancer therapeutic agent to the subject, depending upon the type of disease to be treated or the site of the disease. The anti-cancer therapeutic agent can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, an anti-cancer therapeutic agent may be administered to the subject via injectable depot routes of administration such as using 1-, 3-, or 6-month depot injectable or biodegradable materials and methods.

In one embodiment, an anti-cancer therapeutic agent is administered via site-specific or targeted local delivery techniques. Examples of site-specific or targeted local delivery techniques include various implantable depot sources of the agent or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211 and U.S. Pat. No. 5,981,568, the contents of each of which are incorporated by reference herein for this purpose.

In some embodiments, more than one anti-cancer therapeutic agent, such as an antibody and a small molecule inhibitory compound, may be administered to a subject in need of the treatment. The agents may be of the same type or different types from each other. At least one, at least two, at least three, at least four, or at least five different agents may be co-administered. Generally anti-cancer agents for administration have complementary activities that do not adversely affect each other. Anti-cancer therapeutic agents may also be used in conjunction with other agents that serve to enhance and/or complement the effectiveness of the agents.

Treatment efficacy can be assessed by methods well-known in the art, e.g., monitoring tumor growth or formation in a patient subjected to the treatment. Alternatively, or in addition to, treatment efficacy can be assessed by monitoring tumor type over the course of treatment (e.g., before, during, and after treatment).

In some embodiments, an anti-cancer therapeutic agent is an antibody, an immunotherapy, a radiation therapy, a surgical therapy, and/or a chemotherapy.

Examples of the antibody anti-cancer agents include, but are not limited to, alemtuzumab (Campath), trastuzumab (Herceptin), Ibritumomab tiuxetan (Zevalin), Brentuximab vedotin (Adcetris), Ado-trastuzumab emtansine (Kadcyla), blinatumomab (Blincyto), Bevacizumab (Avastin), Cetuximab (Erbitux), ipilimumab (Yervoy), nivolumab (Opdivo), pembrolizumab (Keytruda), atezolizumab (Tecentriq), avelumab (Bavencio), durvalumab (Imfinzi), and panitumumab (Vectibix).

Examples of an immunotherapy include, but are not limited to, a PD-1 inhibitor or a PD-L1 inhibitor (e.g., nivolumab (Opdivo), pembrolizumab (Keytruda), atezolizumab (Tecentriq), avelumab (Bavencio), durvalumab (Imfinzi)), a CTLA-4 inhibitor, adoptive cell transfer, therapeutic cancer vaccines, oncolytic virus therapy, T-cell therapy, and immune checkpoint inhibitors.

Examples of radiation therapy include, but are not limited to, ionizing radiation, gamma-radiation, neutron beam radiotherapy, electron beam radiotherapy, proton therapy, brachytherapy, systemic radioactive isotopes, and radiosensitizers.

Examples of a surgical therapy include, but are not limited to, a curative surgery (e.g., tumor removal surgery), a preventive surgery, a laparoscopic surgery, and a laser surgery.

Examples of the chemotherapeutic agents include, but are not limited to, Carboplatin or Cisplatin, Docetaxel, Gemcitabine, Nab-Paclitaxel, Paclitaxel, Pemetrexed, and Vinorelbine. Additional examples of chemotherapy include, but are not limited to, Platinating agents, such as Carboplatin, Oxaliplatin, Cisplatin, Nedaplatin, Satraplatin, Lobaplatin, Triplatin, Tetranitrate, Picoplatin, Prolindac, Aroplatin and other derivatives; Topoisomerase I inhibitors, such as Camptothecin, Topotecan, irinotecan/SN38, rubitecan, Belotecan, and other derivatives; Topoisomerase II inhibitors, such as Etoposide (VP-16), Daunorubicin, a doxorubicin agent (e.g., doxorubicin, doxorubicin hydrochloride, doxorubicin analogs, or doxorubicin and salts or analogs thereof in liposomes), Mitoxantrone, Aclarubicin, Epirubicin, Idarubicin, Amrubicin, Amsacrine, Pirarubicin, Valrubicin, Zorubicin, Teniposide and other derivatives; Antimetabolites, such as Folic family (Methotrexate, Pemetrexed, Raltitrexed, Aminopterin, and relatives or derivatives thereof); Purine antagonists (Thioguanine, Fludarabine, Cladribine, 6-Mercaptopurine, Pentostatin, clofarabine, and relatives or derivatives thereof) and Pyrimidine antagonists (Cytarabine, Floxuridine, Azacitidine, Tegafur, Carmofur, Capacitabine, Gemcitabine, hydroxyurea, 5-Fluorouracil (5FU), and relatives or derivatives thereof); Alkylating agents, such as Nitrogen mustards (e.g., Cyclophosphamide, Melphalan, Chlorambucil, mechlorethamine, Ifosfamide, mechlorethamine, Trofosfamide, Prednimustine, Bendamustine, Uramustine, Estramustine, and relatives or derivatives thereof); nitrosoureas (e.g., Carmustine, Lomustine, Semustine, Fotemustine, Nimustine, Ranimustine, Streptozocin, and relatives or derivatives thereof); Triazenes (e.g., Dacarbazine, Altretamine, Temozolomide, and relatives or derivatives thereof); Alkyl sulphonates (e.g., Busulfan, Mannosulfan, Treosulfan, and relatives or derivatives thereof); Procarbazine; Mitobronitol, and Aziridines (e.g., Carboquone, Triaziquone, ThioTEPA, triethylenemalamine, and relatives or derivatives thereof); Antibiotics, such as Hydroxyurea, Anthracyclines (e.g., doxorubicin agent, daunorubicin, epirubicin and relatives or derivatives thereof); Anthracenediones (e.g., Mitoxantrone and relatives or derivatives thereof); Streptomyces family antibiotics (e.g., Bleomycin, Mitomycin C, Actinomycin, and Plicamycin); and ultraviolet light.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize or be able to ascertain using no more than routine experimentation many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The terms “approximately,” “substantially,” and “about” may be used to mean within ±20% of a target value in some embodiments, within ±10% of a target value in some embodiments, within ±5% of a target value in some embodiments, within ±2% of a target value in some embodiments. The terms “approximately,” “substantially,” and “about” may include the target value.

What is claimed is:

1. A method for identifying at least one tertiary lymphoid structure (TLS) in an image of tissue previously-obtained from a subject, the method comprising:
using at least one computer hardware processor to perform:
obtaining a TLS mask indicating, for each particular pixel of multiple pixels of the image, a respective numeric value indicative of a likelihood that the particular pixel is part of the at least one TLS;
processing at least a portion of the image using a trained neural network model to obtain a tumor infiltrating lymphocyte (TIL) mask indicating, for each particular pixel of pixels of at least the portion of the image, a respective numeric value indicative of a likelihood that the particular pixel is part of a TIL, wherein the trained neural network model is trained to predict, for the particular pixel, the respective numeric value indicative of the likelihood that the particular pixel is part of the TIL;
identifying boundaries of the at least one TLS in the image using the TLS mask and the TIL mask; and
identifying one or more characteristics of the at least one TLS using the boundaries of the at least one TLS in the image.

2. The method of claim 1, wherein identifying the boundaries of the at least one TLS in the image comprises:
identifying the boundaries based on an overlap between the TLS mask and the TIL mask, the identifying comprising:
identifying one or more pixels for which the TIL mask indicates a respective one or more numeric values that are greater than or equal to a first threshold and for which the TLS mask indicates a respective one or more numeric values that are greater than or equal to a second threshold.

3. The method of claim 2, wherein identifying the one or more pixels for which the TLS mask indicates the respective one or more numeric values that are greater than or equal to the second threshold comprises:
generating a binary version of the TLS mask; and
identifying contours of at least one portion of the image by applying a border-following algorithm to the binary version of the TLS mask, wherein the one or more pixels are positioned within the contours of the at least one portion of the image.

4. The method of claim 1, further comprising:
obtaining a set of sub-images of the image of the tissue,
wherein processing the image using the trained neural network model to obtain the TIL mask comprises:
processing the set of sub-images using the trained neural network model to obtain a respective set of sub-image masks, each sub-image mask in the set of sub-image masks indicating, for a respective sub-image, a respective numeric value indicative of a likelihood that pixels in the respective sub-image are part of a TIL; and
generating the TIL mask using the set of sub-image masks corresponding to the set of sub-images of the image of the tissue.

5. The method of claim 1, wherein the trained neural network model is a TIL neural network model, and wherein obtaining the TLS mask comprises processing the image using a TLS neural network model to obtain the TLS mask, the method further comprising:
obtaining a set of overlapping sub-images of the image of the tissue,
wherein processing the image using the TLS neural network model to obtain the TLS mask comprises:
processing the set of overlapping sub-images using the TLS neural network model to obtain a respective set of pixel-level sub-image masks, each pixel-level sub-image mask in the set of pixel-level sub-image masks indicating, for each particular pixel of multiple individual pixels in a respective particular sub-image, a respective probability that the particular pixel is part of a TLS; and
generating the TLS mask using the set of pixel-level sub-image masks corresponding to the set of overlapping sub-images of the image of the tissue.

6. The method of claim 1, wherein identifying the boundaries of the at least one TLS in the image comprises:
identifying respective boundaries for a plurality of TLSs in the image; and
applying a filter to the respective boundaries identified for the plurality of TLSs in the tissue to obtain the boundaries of the at least one TLS in the image, the applying comprising, for each particular TLS of the plurality of TLSs:
determining a size of the particular TLS in the image;
determining whether the size is greater than or equal to a threshold; and
filtering out the respective boundaries of the particular TLS after determining that the size is not greater than or equal to the threshold.

7. The method of claim 1, wherein identifying the one or more characteristics of the at least one TLS comprises identifying at least one characteristic selected from the group consisting of: a number of TLSs in at least the portion of the image, the number of TLSs in at least the portion of the image normalized by an area of at least the portion of the image, a total area of TLSs in at least the portion of the image, the total area of the TLSs in at least the portion of the image normalized by the area of at least the portion of the image, median area of TLSs in at least the portion of the image, and the median area of the TLSs in at least the portion of the image normalized by the area of at least the portion of the image.

8. The method of claim 1, further comprising:
identifying boundaries of at least one TIL in the image using the TIL mask; and
identifying one or more characteristics of the at least one TIL using the boundaries of the at least one TIL, the identifying comprising:
determining a total area of TILs in at least the portion of the image and/or
determining the total area of the TILs in at least the portion of the image normalized by an area of at least the portion of the image.

9. The method of claim 1,
wherein identifying the boundaries of the at least one TLS in the image comprises identifying the boundaries of the at least one TLS using the TLS mask, the TIL mask, and at least one other feature in the image,
wherein the at least one other feature in the image comprises at least one feature selected from the group consisting of: tissue in the image, tumor tissue in the image, and non-tumor tissue in the image, and
wherein identifying the one or more characteristics of the at least one TLS comprises identifying a total area of the at least one TLS in the tumor tissue in the image and/or identifying a number of the at least one TLS in the tumor tissue in the image.

10. The method of claim 1,
wherein the image of the tissue is a whole slide image (WSI),
wherein the image is a three-channel image comprising at least 10,000 by 10,000 pixel values per channel, and
wherein the trained neural network model comprises at least 10 million, at least 25 million, at least 50 million, or at least 100 million parameters.

11. The method of claim 1, wherein the trained neural network model comprises:
a deconvolution neural network portion;
an adapter neural network portion having an input coupled to an output of the deconvolution neural network portion; and
a classification neural network portion having an input coupled to an output of the adapter neural network portion.

12. The method of claim 1, wherein the trained neural network model is a TIL neural network model, and wherein obtaining the TLS mask comprises processing the image using a TLS neural network model.

13. The method of claim 12, wherein the TLS neural network model comprises at least 10 million, at least 25 million, at least 50 million, or at least 100 million parameters.

14. The method of claim 13, wherein the TLS neural network model comprises an encoder sub-model, a decoder sub-model, and an auxiliary classifier sub-model.

15. The method of claim 1,
wherein the subject has, is suspected of having, or is at risk of having cancer, and
wherein the cancer is lung adenocarcinoma, breast cancer, cervical squamous cell carcinoma, lung squamous cell carcinoma, head and neck squamous cell carcinoma, gastric adenocarcinoma, colorectal adenocarcinoma, liver adenocarcinoma, pancreatic adenocarcinoma, or melanoma.

16. The method of claim 1, further comprising:
determining, based on the one or more characteristics of the at least one TLS, to administer an immunotherapy to the subject.

17. The method of claim 16, further comprising:
administering the immunotherapy to the subject.

18. The method of claim 17, wherein administering the immunotherapy to the subject comprises administering pembrolizumab, nivolumab, atezolizumab, or durvalumab.

19. A system, comprising:
at least one computer hardware processor; and
at least one non-transitory computer-readable storage medium storing processor-executable instructions that, when executed by the at least one computer hardware processor, cause the at least one computer hardware processor to perform a method for identifying at least one tertiary lymphoid structure (TLS) in an image of tissue previously-obtained from a subject, the method comprising:
obtaining a TLS mask indicating, for each particular pixel of multiple pixels of the image, a respective numeric value indicative of a likelihood that the particular pixel is part of the at least one TLS;
processing at least a portion of the image using a trained neural network model to obtain a tumor infiltrating lymphocyte (TIL) mask indicating, for each particular pixel of pixels of at least the portion of the image, a respective numeric value indicative of a likelihood that the particular pixel is part of a TIL, wherein the trained neural network model is trained to predict, for the particular pixel, the respective numeric value indicative of the likelihood that the particular pixel is part of the TIL;
identifying boundaries of the at least one TLS in the image using the TLS mask and the TIL mask; and
identifying one or more characteristics of the at least one TLS using the boundaries of the at least one TLS in the image.

20. At least one non-transitory computer-readable storage medium storing processor-executable instructions that, when executed by at least one computer hardware processor, cause the at least one computer hardware processor to perform a method for identifying at least one tertiary lymphoid structure (TLS) in an image of tissue previously-obtained from a subject, the method comprising:
obtaining a TLS mask indicating, for each particular pixel of multiple pixels of the image, a respective numeric value indicative of a likelihood that the particular pixel is part of the at least one TLS;
processing at least a portion of the image using a trained neural network model to obtain a tumor infiltrating lymphocyte (TIL) mask indicating, for each particular pixel of pixels of at least the portion of the image, a respective numeric value indicative of a likelihood that the particular pixel is part of a TIL, wherein the trained neural network model is trained to predict, for the particular pixel, the respective numeric value indicative of the likelihood that the particular pixel is part of the TIL;
identifying boundaries of the at least one TLS in the image using the TLS mask and the TIL mask; and
identifying one or more characteristics of the at least one TLS using the boundaries of the at least one TLS in the image.
